(19) United States
(12) Patent Application Publication
(10) Pub. No.: US 2023/0127355 A1
(43) Pub. Date: Apr. 27, 2023

(54) Methods and Systems for Engineering Respiration Rate-Related Features From Biophysical Signals for Use in Characterizing Physiological Systems

(71) Applicant: Analytics for Life Inc., Toronto (CA)

(72) Inventors: Mehdi Paak, Toronto (CA); Timothy William Fawcett Burton, Ottawa (CA); Farhad Fathieh, North York (CA)

(21) Appl. No.: 17/891,224

(22) Filed: Aug. 19, 2022

Related U.S. Application Data
(60) Provisional application No. 63/235,966, filed on Aug. 23, 2021.

(51) Int. Cl. A61B 5/00; A61B 5/145

(52) U.S. Cl. CPC A61B 5/0006; A61B 5/145

(57) ABSTRACT

The exemplified methods and systems (e.g., machine-learned systems) facilitate the use of respiration rate-related features, or parameters, in a model or classifier to estimate metrics associated with the physiological state of a subject, including for the presence or non-presence of a disease, medical condition, or indication of either. The estimated metric may be used to assist a physician or other healthcare provider in diagnosing the presence or non-presence and/or severity and/or localization of diseases, medical conditions, or indication of either or in the treatment of said diseases or indicating conditions. In some cases, such respiration rate-related features are generated from a synthetic respiration waveform that represents, and is used as a proxy to, the true respiration waveform. The synthetic respiration waveform may be used in its own independent diagnostic and/or control applications in some embodiments.

### RELATED APPLICATION

This US application claims priority to, and the benefit of, U.S. Provisional Pat. Application No. 63/235,966, filed Aug. 23, 2021, entitled “Methods and Systems for Engineering Respiration Rate-Related Features from Biophysical Signals for Use in Characterizing Physiological Systems,” which is incorporated by reference herein in its entirety.

### FIELD OF THE INVENTIONS

The present disclosure generally relates to methods and systems for engineering features or parameters from biophysical signals for use in diagnostic applications; in particular, the engineering and use of respiration rate-related features, some of which may be based on a proxy respiration waveform, for use in characterizing one or more physiological systems and their associated functions, activities, and abnormalities. The features or parameters may also be used for monitoring or tracking, controls of medical equipment, or to guide the treatment of a disease, medical condition, or an indication of either.

### BACKGROUND

There are numerous methods and systems for assisting a healthcare professional in diagnosing disease. Some of these involve the use of invasive or minimally invasive techniques, radiation, exercise or stress, or pharmacological agents, sometimes in combination, with their attendant risks and other disadvantages.

Diastolic heart failure, a major cause of morbidity and mortality, is defined as symptoms of heart failure in a patient with preserved left ventricular function. It is characterized by a stiff left ventricle with decreased compliance and impaired relaxation leading to increased end-diastolic pressure in the left ventricle, which is measured through left heart catheterization. Current clinical standard of care for diagnosing pulmonary hypertension (PH), and for pulmonary arterial hypertension (PAH), in particular, involves a cardiac catheterization of the right side of the heart that directly measures the pressure in the pulmonary arteries. Coronary angiography is the current standard of care used to assess coronary arterial disease (CAD) as determined through the coronary lesions described by a treating physician. Non-invasive imaging systems such as magnetic resonance imaging and computed tomography require specialized facilities to acquire images of blood flow and arterial blockages of a patient that are reviewed by radiologists.

It is desirable to have a system that can assist healthcare professionals in the diagnosis of cardiac disease and various other diseases and conditions without the aforementioned disadvantages.

### SUMMARY

A clinical evaluation system and method are disclosed that facilitate the use of one or more respiration rate-related features or parameters determined from biophysical signals such as cardiac/biopotential signals and/or photoplethysmography signals that are acquired, in preferred embodiments, non-invasively from surface sensors placed on a patient while the patient is at rest. The respiration rate-related features or parameters can be used in a model or classifier (e.g., a machine-learned classifier) to estimate metrics associated with the physiological state of a patient, including for the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions.

The estimation or determined likelihood of the presence or non-presence of a disease, condition, or indication of either can supplant, augment, or replace other evaluation or measurement modalities for the assessment of a disease or medical condition. In some cases, a determination can take the form of a numerical score and related information.

Examples of respiration rate-related features or parameters include measures that are derived based on (i) heart rate variability information, (ii) respiration rate information, (iii) an assessed complexity (e.g., relative entropy) between one or more input modulated signals associated with respiration and a baseline modulated signal, (iv) an assessed maximum mean discrepancy among calculated distances determined between an estimated power of a synthetic respiration waveform and estimated powers of one or more input modulated signals associated with respiration, and (v) assessed cross-spectral agreement between a synthetic respiration waveform and one or more input modulated signals associated with respiration. The respiration rate-related features or parameters may include statistical or geometric properties (e.g., mean, skew, kurtosis, standard derivation) of distributions of these various measures. Respiration rate-related features or parameters and classes of respiration rate-related features, as later disclosed herein, were developed in the context of a machine learning system for diagnostic-assisting applications, though they may be broadly applied in treatment, controls, monitoring, or tracking applications.

As used herein, the term “feature” (in the context of machine learning and pattern recognition and as used herein) generally refers to an individual measurable property or characteristic of a phenomenon being observed. A feature is defined by analysis and may be determined in groups in combination with other features from a common model or analytical framework.

As used herein, “metric” refers to an estimation or likelihood of the presence, non-presence, severity, and/or localization (where applicable) of one or more diseases, conditions, or indication(s) of either, in a physiological system or systems. Notably, the exemplified methods and systems can be used in certain embodiments described herein to acquire biophysical signals and/or to otherwise collect data from a patient and to evaluate those signals and/or data in signal processing and classifier operations to evaluate for a disease, condition, or indicator of one that can supplant, augment, or replace other evaluation modalities via one or more metrics. In some cases, a metric can take the form of a numerical score and related information.

In the context of cardiovascular and respiratory systems, examples of diseases and conditions to which such metrics can relate include, for example: (i) heart failure (e.g., left-side or right-side heart failure; heart failure with preserved ejection fraction (HFpEF)), (ii) coronary artery disease (CAD), (iii) various forms of pulmonary hypertension (PH) including without limitation pulmonary arterial hypertension (PAH), (iv) abnormal left ventricular ejection fraction (LVEF), and various other diseases or conditions. An example indicator of certain forms of heart failure is the presence or non-presence of elevated or abnormal left-ventricular end-diastolic pressure (LVEDP). An example indicator of certain forms of pulmonary hypertension is the presence or non-presence of elevated or abnormal mean pulmonary arterial pressure (mPAP).

In some cases, the respiration rate-related features are generated from a synthetic respiration waveform that represents, and is used as a proxy to the true respiration waveform. The synthetic respiration waveform and various parameters disclosed herein may be used in their own independent diagnostics, treatment, controls, monitoring, and/or tracking applications.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments and, together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 is a schematic diagram of example modules, or components, configured to non-invasively compute respiration rate-related features or parameters to generate one or more metrics associated with the physiological state of a patient in accordance with an illustrative embodiment.

FIG. 2 shows an example biophysical signal capture system or component and its use in non-invasively collecting biophysical signals of a patient in a clinical setting in accordance with an illustrative embodiment.

FIGS. 3A-3C each shows an example method to use respiration rate-related features/parameters or their intermediate data in a practical application for diagnostics, treatment, monitoring, or tracking.

FIG. 4 shows an example schematic diagram of functional relationships between the respiratory system and the biophysical signals non-invasively acquired through the biophysical signal capture system of FIG. 2 in accordance with an illustrative embodiment.

FIGS. 5-9 each shows an example respiration rate-related feature computation module configured to determine values of respiration rate-related features or parameters in accordance with an illustrative embodiment. One or more features generated from any one of these modules may be used to generate the one or more metrics associated with the physiological state of a patient.

FIG. 10 shows a detailed implementation of a respiration rate feature computation module of FIG. 5 in accordance with an illustrative embodiment.

FIG. 11 shows a detailed implementation of a heart-rate variability feature computation module of FIG. 6 in accordance with an illustrative embodiment.

FIG. 12 shows a detailed implementation of a relative-entropy associated feature computation module of FIG. 7 in accordance with an illustrative embodiment.

FIGS. 13A and 13B show a detailed implementation of a maximum mean discrepancy associated feature computation module of FIG. 8 in accordance with an illustrative embodiment.

FIG. 14 shows a detailed implementation of a coherence-associated feature computation module of FIG. 9 in accordance with an illustrative embodiment.

FIG. 15A shows a schematic diagram of an example clinical evaluation system configured to use respiration rate-related features among other computed features to generate one or more metrics associated with the physiological state of a patient in accordance with an illustrative embodiment.

FIG. 15B shows a schematic diagram of the operation of the example clinical evaluation system of FIG. 15A in accordance with an illustrative embodiment.

### DETAILED DESCRIPTION

Each and every feature described herein, and each and every combination of two or more of such features, is included within the scope of the present invention provided that the features included in such a combination are not mutually inconsistent.

While the present disclosure is directed to the practical assessment of biophysical signals, e.g., raw or pre-processed photoplethysmographic signals, biopotential/cardiac signals, etc., in the diagnosis, tracking, and treatment of cardiac-related pathologies and conditions, such assessment can be applied to the diagnosis, tracking, and treatment (including without limitation surgical, minimally invasive, lifestyle, nutritional, and/or pharmacologic treatment, etc.) of any pathologies or conditions in which a biophysical signal is involved in any relevant system of a living body. The assessment may be used in the controls of medical equipment or wearable devices or in monitoring applications (e.g., to report respiration rate or associated waveforms generated using the biophysical signals as disclosed therein).

The terms “subject” and “patient” as used herein are generally used interchangeably to refer to those who had undergone analysis performed by the exemplary systems and methods.

The term “cardiac signal” as used herein refers to one or more signals directly or indirectly associated with the structure, function, and/or activity of the cardiovascular system –including aspects of that signal’s electrical/electrochemical conduction – that, e.g., cause contraction of the myocardium. A cardiac signal may include, in some embodiments, biopotential signals or electrocardiographic signals, e.g., those acquired via an electrocardiogram (ECG), the cardiac and photoplethysmographic waveform or signal capture or recording instrument later described herein, or other modalities.

The term “biophysical signal” as used herein includes but is not limited to one or more cardiac signal(s), neurological signal(s), ballistocardiographic signal(s), and/or photoplethysmographic signal(s), but it also encompasses more broadly any physiological signal from which information may be obtained. Not intending to be limited by example, one may classify biophysical signals into types or categories that can include, for example, electrical (e.g., certain cardiac and neurological system-related signals that can be observed, identified, and/or quantified by techniques such as the measurement of voltage/potential (e.g., biopotential), impedance, resistivity, conductivity, current, etc. in various domains such as time and/or frequency), magnetic, electromagnetic, optical (e.g., signals that can be observed, identified and/or quantified by techniques such as reflectance, interferometry, spectroscopy, absorbance, transmissivity, visual observation, photoplethysmography, and the like), acoustic, chemical, mechanical (e.g., signals related to fluid flow, pressure, motion, vibration, displacement, strain), thermal, and electrochemical (e.g., signals that can be correlated to the presence of certain analytes, such as glucose). Biophysical signals may in some cases be described in the context of a physiological system (e.g., respiratory, circulatory (cardiovascular, pulmonary), nervous, lymphatic, endocrine, digestive, excretory, muscular, skeletal, renal/urinary/excretory, immune, integumentary/exocrine and reproductive systems), one or more organ system(s) (e.g., signals that may be unique to the heart and lungs as they work together), or in the context of tissue (e.g., muscle, fat, nerves, connective tissue, bone), cells, organelles, molecules (e.g., water, proteins, fats, carbohydrates, gases, free radicals, inorganic ions, minerals, acids, and other compounds, elements, and their subatomic components. Unless stated otherwise, the term “biophysical signal acquisition” generally refers to any passive or active means of acquiring a biophysical signal from a physiological system, such as a mammalian or non-mammalian organism. Passive and active biophysical signal acquisition generally refers to the observation of natural or induced electrical, magnetic, optical, and/or acoustics emittance of the body tissue. Non-limiting examples of passive and active biophysical signal acquisition means include, e.g., voltage/potential, current, magnetic, optical, acoustic, and other non-active ways of observing the natural emittance of the body tissue, and in some instances, inducing such emittance. Non-limiting examples of passive and active biophysical signal acquisition means include, e.g., ultrasound, radio waves, microwaves, infrared and/or visible light (e.g., for use in pulse oximetry or photoplethysmography), visible light, ultraviolet light, and other ways of actively interrogating the body tissue that does not involve ionizing energy or radiation (e.g., X-ray). An active biophysical signal acquisition may involve excitation-emission spectroscopy (including, for example, excitation-emission fluorescence). The active biophysical signal acquisition may also involve transmitting ionizing energy or radiation (e.g., X-ray) (also referred to as “ionizing biophysical signal”) to the body tissue. Passive and active biophysical signal acquisition means can be performed in conjunction with invasive procedures (e.g., via surgery or invasive radiologic intervention protocols) or non-invasively (e.g., via imaging, ablation, heart contraction regulation (e.g., via pacemakers), catheterization, etc.).

The term “photoplethysmographic signal” as used herein refers to one or more signals or waveforms acquired from optical sensors that correspond to measured changes in light absorption by oxygenated and deoxygenated hemoglobin, such as light having wavelengths in the red and infrared spectra. Photoplethysmographic signal(s), in some embodiments, include a raw signal(s) acquired via a pulse oximeter or a photoplethysmogram (PPG). In some embodiments, photoplethysmographic signal(s) are acquired from off-the-shelf, custom, and/or dedicated equipment or circuitries that are configured to acquire such signal waveforms for the purpose of monitoring health and/or diagnosing disease or abnormal conditions. The photoplethysmographic signal(s) typically include a red photoplethysmographic signal (e.g., an electromagnetic signal in the visible light spectrum most dominantly having a wavelength of approximately 625 to 740 nanometers) and an infrared photoplethysmographic signal (e.g., an electromagnetic signal extending from the nominal red edge of the visible spectrum up to about 1 mm), though other spectra such as near-infrared, blue and green may be used in different combinations, depending on the type and/or mode of PPG being employed.

The term “ballistocardiographic signal,” as used herein, refers to a signal or group of signals that generally reflect the flow of blood through the entire body that may be observed through vibration, acoustic, movement, or orientation. In some embodiments, ballistocardiographic signals are acquired by wearable devices, such as vibration, acoustic, movement, or orientation-based seismocardiogram (SCG) sensors, which can measure the body’s vibrations or orientation as recorded by sensors mounted close to the heart. Seismocardiogram sensors are generally used to acquire “seismocardiogram,” which is used interchangeably with the term “ballistocardiogram” herein. In other embodiments, ballistocardiographic signals may be acquired by external equipment, e.g., bed or surface-based equipment that measures phenomena such as a change in body weight as blood moves back and forth in the longitudinal direction between the head and feet. In such embodiments, the volume of blood in each location may change dynamically and be reflected in the weight measured at each location on the bed as well as the rate of change of that weight.

In addition, the methods and systems described in the various embodiments herein are not so limited and may be utilized in any context of another physiological system or systems, organs, tissue, cells, etc., of a living body. By way of example only, two biophysical signal types that may be useful in the cardiovascular context include cardiac/biopotential signals that may be acquired via conventional electrocardiogram (ECG/EKG) equipment, bipolar wide-band biopotential (cardiac) signals that may be acquired from other equipment such as those described herein, and signals that may be acquired by various plethysmographic techniques, such as, e.g., photoplethysmography. In another example, the two biophysical signal types can be further augmented by ballistocardiographic techniques.

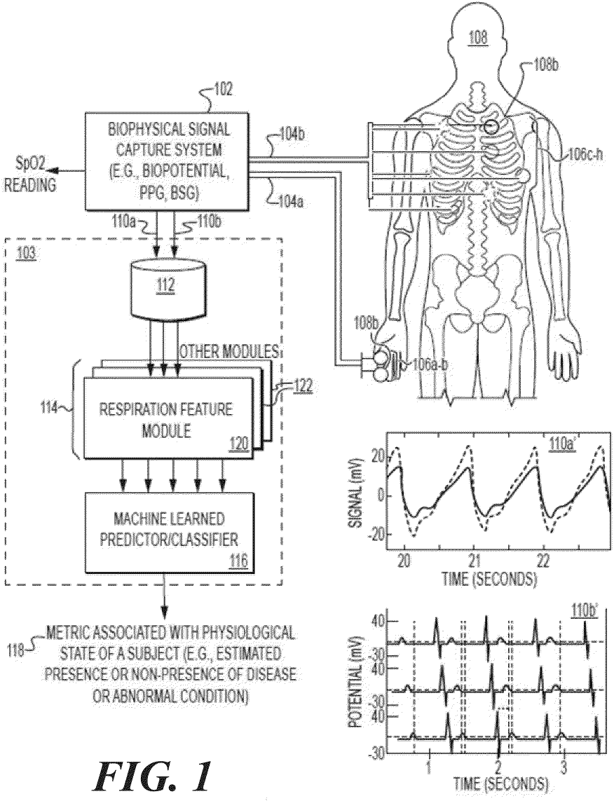

FIG. 1 is a schematic diagram of example modules, or components, configured to non-invasively compute respiration rate-related features or parameters to generate, via a classifier (e.g., machine-learned classifier), one or more metrics associated with the physiological state of a patient in accordance with an illustrative embodiment. The modules or components may be used in a production application or the development of the respiration rate-related features and other classes of features.

The example analysis and classifiers described herein may be used to assist a healthcare provider in the diagnosis and/or treatment of cardiac- and cardiopulmonary-related pathologies and medical conditions, or an indicator of one. Examples include significant coronary artery disease (CAD), one or more forms of heart failure such as, e.g., heart failure with preserved ejection fraction (HFpEF), congestive heart failure, various forms of arrhythmia, valve failure, various forms of pulmonary hypertension, among various other disease and conditions disclosed herein.

In addition, there exist possible indicators of a disease or condition, such as an elevated or abnormal left ventricular end-diastolic pressure (LVEDP) value as it relates to some forms of heart failure, abnormal left ventricular ejection fraction (LVEF) values as they relate to some forms of heart failure or an elevated mean pulmonary arterial pressure (mPAP) value as it relates to pulmonary hypertension and/or pulmonary arterial hypertension. Indicators of the likelihood that such indicators are abnormal/elevated or normal, such as those provided by the example analysis and classifiers described herein, can help a healthcare provider assess or diagnose that the patient has or does not have a given disease or condition. In addition to these metrics associated with a disease state of condition, other measurements and factors may be employed by a healthcare professional in making a diagnosis, such as the results of a physical examination and/or other tests, the patient’s medical history, current medications, etc. The determination of the presence or non-presence of a disease state or medical condition can include the indication (or a metric of measure that is used in the diagnosis) for such disease.

In FIG. 1, the components include at least one non-invasive biophysical signal recorder or capture system 102 and an assessment system 103 that is located, for example, in a cloud or remote infrastructure or in a local system. Biophysical signal capture system 102 (also referred to as a biophysical signal recorder system), in this embodiment, is configured to, e.g., acquire, process, store and transmit synchronously acquired patient’s electrical and hemodynamic signals as one or more types of biophysical signals 104. In the example of FIG. 1, the biophysical signal capture system 102 is configured to synchronously capture two types of biophysical signals shown as first biophysical signals 104a (e.g., synchronously acquired to other first biophysical signals) and second biophysical signals 104b (e.g., synchronously acquired to the other biophysical signals) acquired from measurement probes 106 (e.g., shown as probes 106a and 106b, e.g., comprising hemodynamic sensors for hemodynamic signals 104a, and probes 106c-106h comprising leads for electrical/cardiac signals 104b). The probes 106a-h are placed on, e.g., by being adhered to or placed next to, a surface tissue of a patient 108 (shown at patient locations 108a and 108b). The patient is preferably a human patient, but it can be any mammalian patient. The acquired raw biophysical signals (e.g., 106a and 106b) together form a biophysical-signal data set 110 (shown in FIG. 1 as a first biophysical-signal data set 110a and a second biophysical-signal data set 110b, respectively) that may be stored, e.g., as a single file, preferably, that is identifiable by a recording/signal captured number and/or by a patient’s name and medical record number.

In the FIG. 1 embodiment, the first biophysical-signal data set 110a comprises a set of raw photoplethysmographic, or hemodynamic, signal(s) associated with measured changes in light absorption of oxygenated and/or deoxygenated hemoglobin from the patient at location 108a, and the second biophysical-signal data set 110b comprises a set of raw cardiac or biopotential signal(s) associated with electrical signals of the heart. Though in FIG. 1, raw photoplethysmographic or hemodynamic signal(s) are shown being acquired at a patient’s finger, the signals may be alternatively acquired at the patient’s toe, wrist, forehead, earlobe, neck, etc. Similarly, although the cardiac or biopotential signal(s) are shown to be acquired via three sets of orthogonal leads, other lead configurations may be used (e.g., 11 lead configuration, 12 lead configuration, etc.).

Plots 110a′ and 110b′ show examples of the first biophysical-signal data set 110a and the second biophysical-signal data set 110a, respectively. Specifically, Plot 110a′ shows an example of an acquired photoplethysmographic or hemodynamic signal. In Plot 110a′, the photoplethysmographic signal is a time series signal having a signal voltage potential as a function of time as acquired from two light sources (e.g., infrared and red-light source). Plot 110b′ shows an example cardiac signal comprising a 3-channel potential time series plot. In some embodiments, the biophysical signal capture system 102 preferably acquires biophysical signals via non-invasive means or component(s). In alternative embodiments, invasive or minimally-invasively means or component(s) may be used to supplement or as substitutes for the non-invasive means (e.g., implanted pressure sensors, chemical sensors, accelerometers, and the like). In still further alternative embodiments, non-invasive and non-contact probes or sensors capable of collecting biophysical signals may be used to supplement or as substitutes for the non-invasive and/or invasive/minimally invasive means, in any combination (e.g., passive thermometers, scanners, cameras, x-ray, magnetic, or other means of non-contact or contact energy data collection system as discussed herein). Subsequent to signal acquisitions and recording, the biophysical signal capture system 102 then provides, e.g., sending over a wireless or wired communication system and/or a network, the acquired biophysical-signal data set 110 (or a data set derived or processed therefrom, e.g., filtered or pre-processed data) to a data repository 112 (e.g., a cloud-based storage area network) of the assessment system 103. In some embodiments, the acquired biophysical-signal data set 110 is sent directly to the assessment system 103 for analysis or is uploaded to a data repository 112 through a secure clinician’s portal.

Biophysical signal capture system 102 is configured with circuitries and computing hardware, software, firmware, middleware, etc., in some embodiments, to acquire, store, transmit, and optionally process both the captured biophysical signals to generate the biophysical-signal data set 110. An example biophysical signal capture system 102 and the acquired biophysical-signal set data 110 are described in U.S. Pat. No. 10,542,898, entitled “Method and Apparatus for Wide-Band Phase Gradient Signal Acquisition,” or U.S. Pat. Publication No. 2018/0249960, entitled “Method and Apparatus for Wide-Band Phase Gradient Signal Acquisition,” each of which is hereby incorporated by reference herein in its entirety.

In some embodiments, biophysical signal capture system 102 includes two or more signal acquisition components, including a first signal acquisition component (not shown) to acquire the first biophysical signals (e.g., photoplethysmographic signals) and includes a second signal acquisition component (not shown) to acquire the second biophysical signals (e.g., cardiac signals). In some embodiments, the electrical signals are acquired at a multi-kilohertz rate for a few minutes, e.g., between 1 kHz and 10 kHz. In other embodiments, the electrical signals are acquired between 10 kHz and 100 kHz. The hemodynamic signals may be acquired, e.g., between 100 Hz and 1 kHz.

Biophysical signal capture system 102 may include one or more other signal acquisition components (e.g., sensors such as mechano-acoustic, ballistographic, ballistocardiographic, etc.) for acquiring signals. In other embodiments of the signal capture system 102, a signal acquisition component comprises conventional electrocardiogram (ECG/EKG) equipment (e.g., Holter device, 12 lead ECG, etc.).

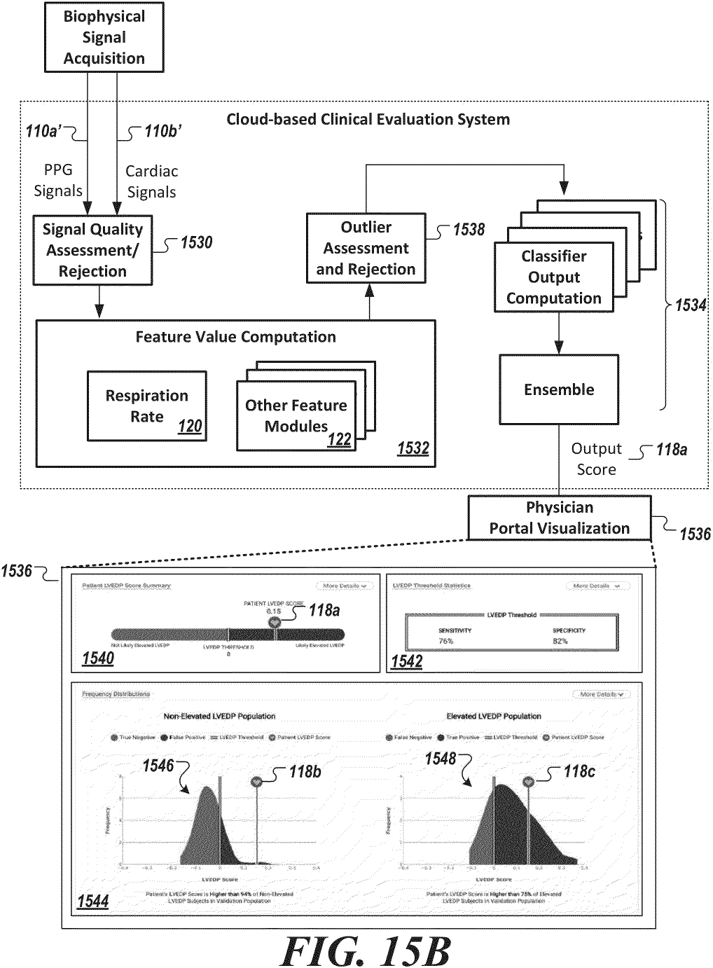

Assessment system 103 comprises, in some embodiments, the data repository 112 and an analytical engine or analyzer (not shown - see FIGS. 15A and 15B). Assessment system 103 may include feature modules 114 and a classifier module 116 (e.g., an ML classifier module). In FIG. 1, Assessment system 103 is configured to retrieve the acquired biophysical signal data set 110, e.g., from the data repository 112, and use it in the feature modules 114, which is shown in FIG. 1 to include a respiration feature module 120 and other modules 122 (later described herein). The features modules 114 compute values of features or parameters, including those of respiration rate-related features, to provide to the classifier module 116, which computes an output 118, e.g., an output score, of the metrics associated with the physiological state of a patient (e.g., an indication of the presence or non-presence of a disease state, medical condition, or an indication of either). Output 118 is subsequently presented, in some embodiments, at a healthcare physician portal (not shown - see FIGS. 15A and 15B) to be used by healthcare professionals for the diagnosis and treatment of pathology or a medical condition. In some embodiments, a portal may be configured (e.g., tailored) for access by, e.g., patients, caregivers, researchers, etc., with output 118 configured for the portal’s intended audience. Other data and information may also be a part of output 118 (e.g., the acquired biophysical signals or other patient’s information and medical history).

Classifier module 116 (e.g., ML classifier module) may include transfer functions, look-up tables, models, or operators developed based on algorithms such as but not limited to decision trees, random forests, neural networks, linear models, Gaussian processes, nearest neighbor, SVMs, Naive Bayes, etc. In some embodiments, classifier module 116 may include models that are developed based on ML techniques described in U.S. Provisional Pat. Application no. 63/235,960, filed Aug. 23, 2021, entitled “Method and System to Non-Invasively Assess Elevated Left Ventricular End-Diastolic Pressure”; U.S. Pat. Publication No. 20190026430, entitled “Discovering Novel Features to Use in Machine Learning Techniques, such as Machine Learning Techniques for Diagnosing Medical Conditions”; or U.S. Pat. Publication No. 20190026431, entitled “Discovering Genomes to Use in Machine Learning Techniques,” each of which is hereby incorporated by reference herein in its entirety.

### Example Biophysical Signal Acquisition

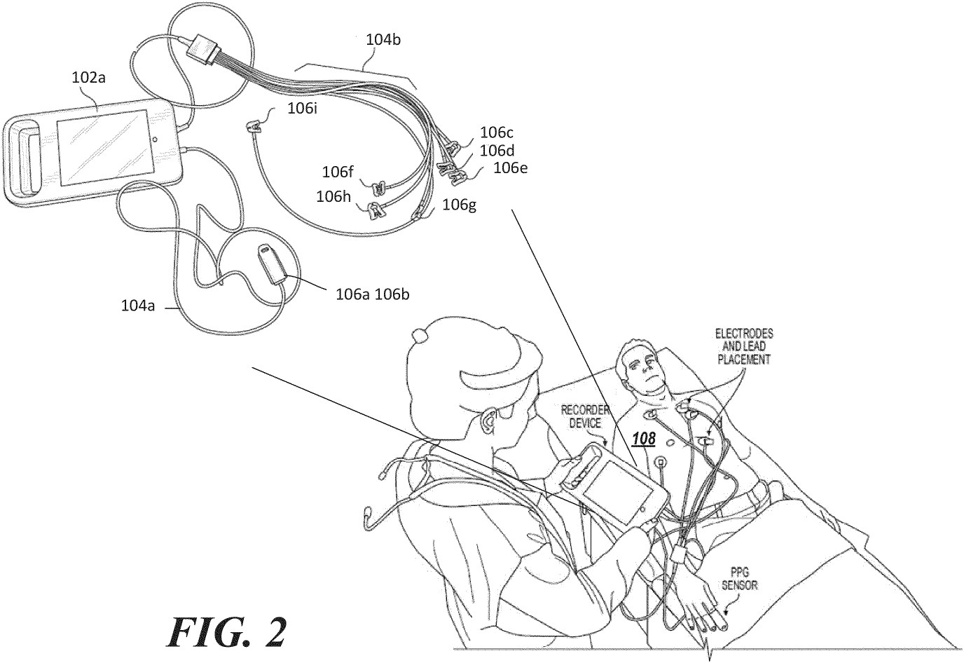

FIG. 2 shows a biophysical signal capture system 102 (shown as 102a) and its use in non-invasively collecting biophysical signals of a patient in a clinical setting in accordance with an illustrative embodiment. In FIG. 2, the biophysical signal capture system 102a is configured to capture two types of biophysical signals from the patient 108 while the patient is at rest. The biophysical signal capture system 102a synchronously acquires the patient’s (i) electrical signals (e.g., cardiac signals corresponding to the second biophysical-signal data set 110b) from the torso using orthogonally placed sensors (106c-106h; 106i is a 7^{th} common-mode reference lead) and (ii) hemodynamic signals (e.g., PPG signals corresponding to the first biophysical-signal data set 110a) from the finger using a photoplethysmographic sensor (e.g., collecting signals 106a, 106b).

As shown in FIG. 2, the electrical and hemodynamic signals (e.g., 104a, 104b) are passively collected via commercially available sensors applied to the patient’s skin. The signals may be acquired beneficially without patient exposure to ionizing radiation or radiological contrast agents and without patient exercise or the use of pharmacologic stressors. The biophysical signal capture system 102a can be used in any setting conducive for a healthcare professional, such as a technician or nurse, to acquire the requisite data and where a cellular signal or Wi-Fi connection can be established.

The electrical signals (e.g., corresponding to the second biophysical signal data set 110b) are collected using three orthogonally paired surface electrodes arranged across the patient’s chest and back along with a reference lead. The electrical signals are acquired, in some embodiments, using a low-pass anti-aliasing filter (e.g., ~ 2 kHz) at a multi-kilohertz rate (e.g., 8 thousand samples per second for each of the six channels) for a few minutes (e.g., 215 seconds). In alternative embodiments, the biophysical signals may be continuously/intermittently acquired for monitoring, and portions of the acquired signals are used for analysis. The hemodynamic signals (e.g., corresponding to the first biophysical signal data set 110a) are collected using a photoplethysmographic sensor placed on a finger. The photo-absorption of red light (e.g., any wavelengths between 600-750 nm) and infrared light (e.g., any wavelengths between 850-950 nm) are recorded, in some embodiments, at a rate of 500 samples per second over the same period. The biophysical signal capture system 102a may include a common mode drive that reduces common-mode environmental noise in the signal. The photoplethysmographic and cardiac signals were simultaneously acquired for each patient. Jitter (inter-modality jitter) in the data may be less than about 10 microseconds µs). Jitter among the cardiac signal channels may be less than 10 microseconds, e.g., around ten femtoseconds (fs).

A signal data package containing the patient metadata and signal data may be compiled at the completion of the signal acquisition procedure. This data package may be encrypted before the biophysical signal capture system 102a transfers the package to the data repository 112. In some embodiments, the data package is transferred to the assessment system (e.g., 103). The transfer is initiated, in some embodiments, following the completion of the signal acquisition procedure without any user intervention. The data repository 112 is hosted, in some embodiments, on a cloud storage service that can provide secure, redundant, cloud-based storage for the patient’s data packages, e.g., Amazon Simple Storage Service (i.e., “Amazon S3”). The biophysical signal capture system 102a also provides an interface for the practitioner to receive notification of an improper signal acquisition to alert the practitioner to immediately acquire additional data from the patient.

### Example Method of Operation

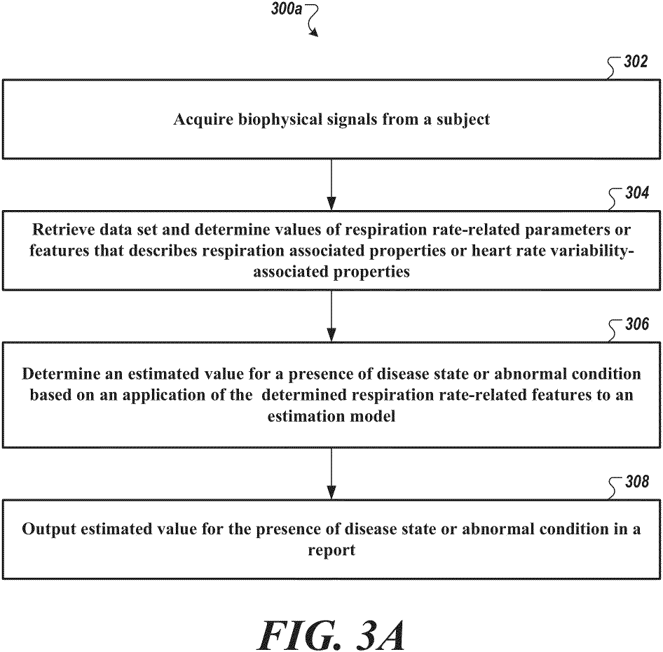

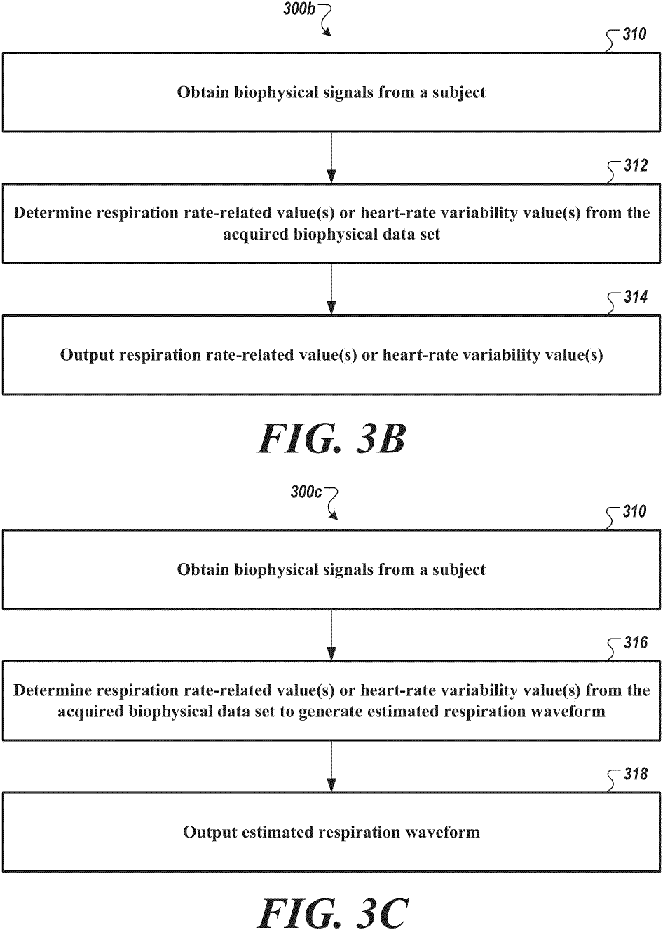

FIGS. 3A-3C each shows an example method to use respiration rate-related features or their intermediate outputs in a practical application for diagnostics, treatment, monitoring, or tracking.

Estimation of Presence of Disease State or Indicating Condition. FIG. 3A shows a method 300a that employs respiration rate-related parameters or features to determine estimators of the presence of a disease state, medical condition, or indication of either, e.g., to aid in the diagnosis, tracking, or treatment. Method 300a includes the step of acquiring (302) biophysical signals from a patient (e.g., cardiac signals, photoplethysmographic signals, ballistocardiographic signals), e.g., as described in relation to FIGS. 1 and 2 and other examples as described herein. In some embodiments, the acquired biophysical signals are transmitted for remote storage and analysis. In other embodiments, the acquired biophysical signals are stored and analyzed locally.

As stated above, one example in the cardiac context is the estimation of the presence of abnormal left-ventricular end-diastolic pressure (LVEDP) or mean pulmonary artery pressure (mPAP), significant coronary artery disease (CAD), abnormal left ventricular ejection fraction (LVEF), and one or more forms of pulmonary hypertension (PH), such as pulmonary arterial hypertension (PAH). Other pathologies or indicating conditions that may be estimated include, e.g., one or more forms of heart failure such as, e.g., heart failure with preserved ejection fraction (HFpEF), arrhythmia, congestive heart failure, valve failure, among various other diseases and medical conditions disclosed herein.

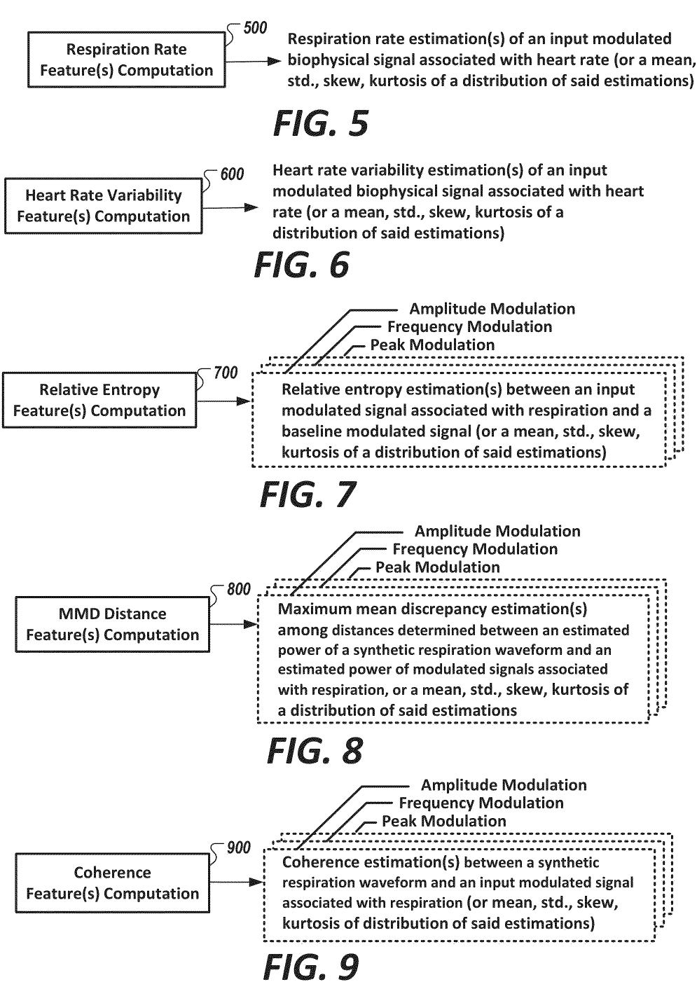

Method 300a further includes the step of retrieving (304) the data set and determining values of respiration rate-related features that describe respiration-associated properties or heart rate variability-associated properties. Example operations to determine the values of respiration rate-related features are provided in relation to FIGS. 5-14 later discussed herein. Method 300a further includes the step of determining (306) an estimated value for a presence of a disease state, medical condition, or an indication of either based on an application of the determined respiration rate-related features to an estimation model (e.g., ML models). An example implementation is provided in relation to FIGS. 15A and 15B.

Method 300a further includes the step of outputting (308) estimated value(s) for the presence of disease state or abnormal condition in a report (e.g., to be used diagnosis or treatment of the disease state, medical condition, or indication of either), e.g., as described in relation to FIGS. 1, 15A, and 15B and other examples described herein.

Diagnostics or Condition Monitoring or Tracking using Estimated Respiration Rate. FIG. 3B shows a method 300b that employs respiration rate-related parameters or features for the monitoring respiration or controls of medical equipment or health monitoring device. Method 300b includes the step of obtaining (302) biophysical signals from a patient (e.g., cardiac signals, photoplethysmographic signals, ballistocardiographic signals, etc.). The operation may be performed continuously or intermittently, e.g., to provide output for a report or as controls for the medical equipment or the health monitoring device.

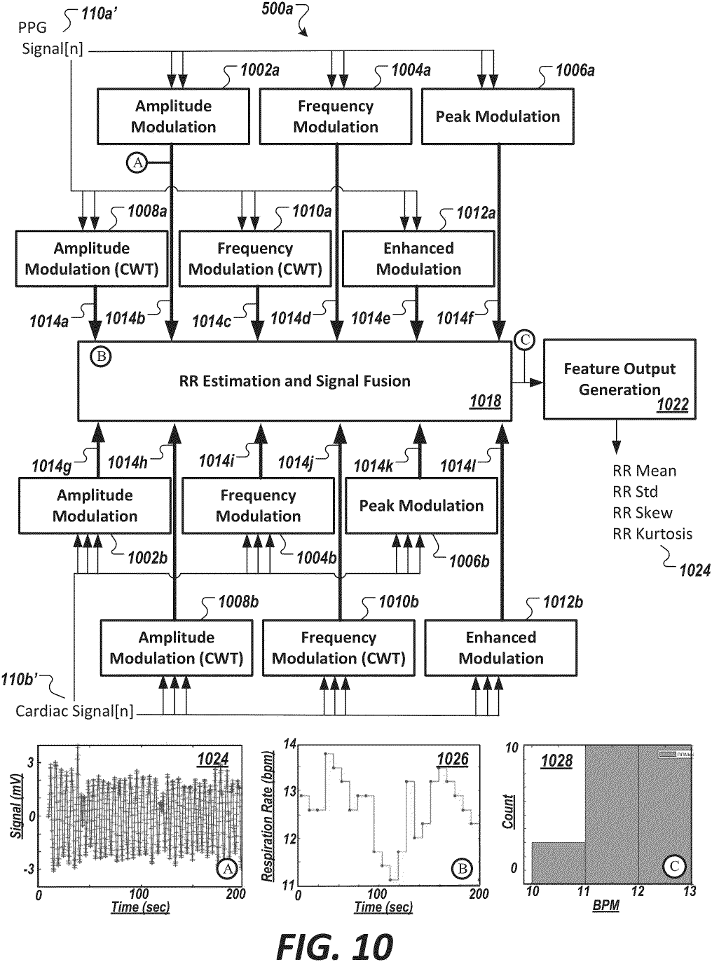

Method 300b further includes determining (310) respiration rate-related value(s) or heart-rate variability value(s) from the acquired biophysical data set, e.g., as described in relation to FIGS. 5-14, such as in FIG. 10.

The method 300b further includes outputting (312) respiration rate-related value(s) or heart-rate variability value(s) (e.g., in a report for use in diagnostics or as signals for controls). For monitoring and tracking, the output may be via a wearable device, a handheld device, or medical diagnostic equipment (e.g., pulse oximeter system, wearable health monitoring systems) to provide augmented data associated with respiration rate or quality of respiration. In some embodiments, the outputs may be used in resuscitation systems, cardiac or pulmonary stress test equipment, pacemakers, etc., in which respiration rate or heart-rate variability is desired.

Diagnostics or Condition Monitoring or Tracking using Estimated Respiration Waveform. FIG. 3C shows a method 300c that employs respiration rate-related parameters or features to generate an estimated respiration waveform for monitoring or tracking of respiration. Method 300b includes the step of obtaining (310) biophysical signals from a patient (e.g., cardiac signals, photoplethysmographic signals, ballistocardiographic signals). The operation may be performed continuously or intermittently, e.g., to provide output for a report or as controls for medical equipment.

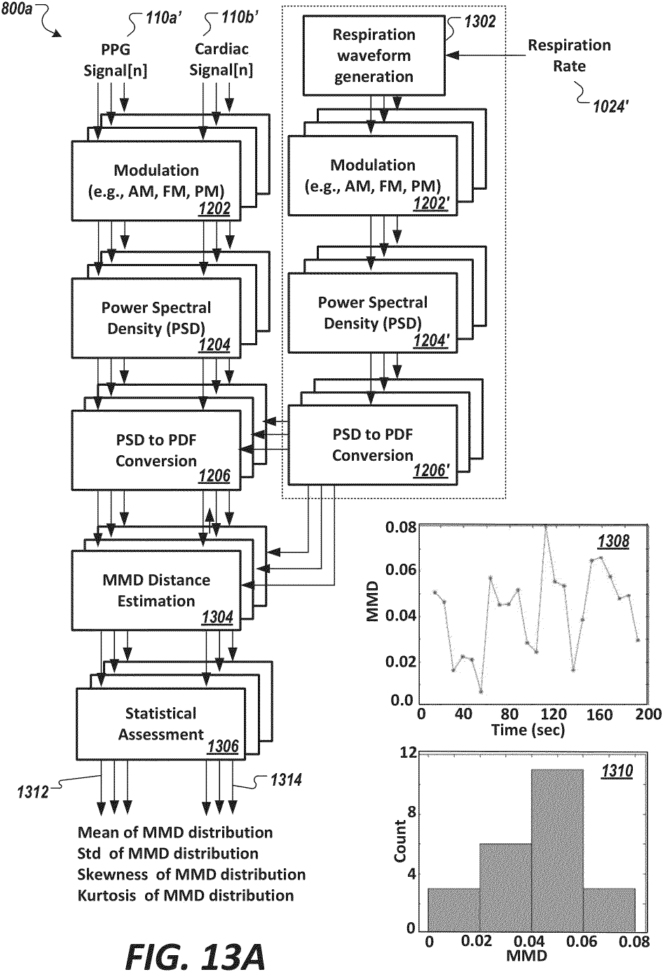

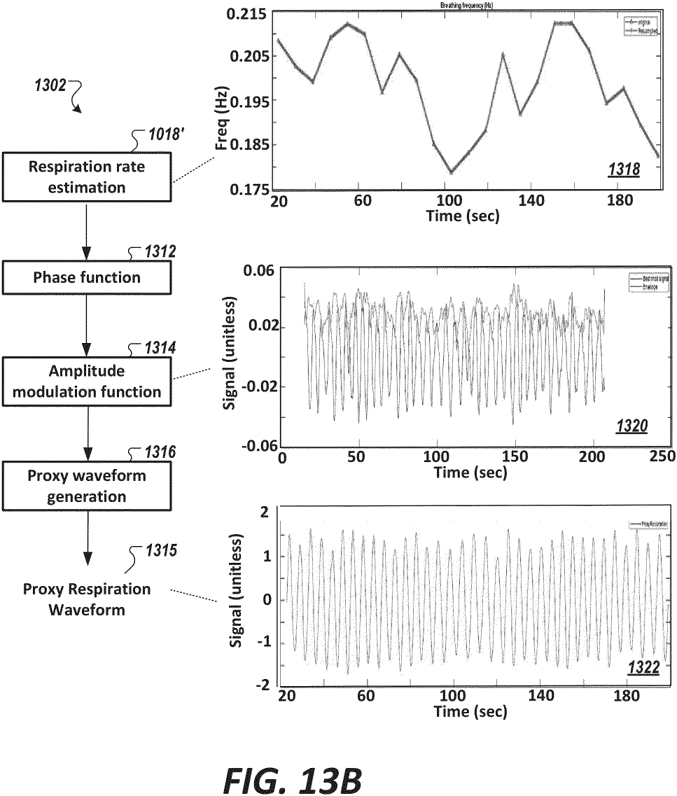

The method 300c includes determining (312) a respiration waveform, e.g., as described in relation to FIGS. 13A and 13B. Method 300c further includes outputting (318) the respiration waveform (e.g., in a report for use in diagnostics or as signals for controls). For monitoring and tracking, the output may be via a wearable device, a handheld device, or medical diagnostic equipment (e.g., pulse oximeter system, wearable health monitoring systems) to provide augmented data associated with respiration waveform. In some embodiments, the outputs may be used in resuscitation systems, cardiac or pulmonary stress test equipment, pacemakers, or other equipment or application in which respiration waveform is desired.

### Respiration Rate-Related Features

In the embodiment of FIG. 1, various features or parameters (as embodied in modules 120 and 122) are used by the assessment system 103 (e.g., comprising an analytical engine or analyzer) to generate one or more metrics associated with the physiological state of a patient, including respiration rate-related features or parameters. Numerous examples of respiration rate-related properties are disclosed herein, including features to five different classes or families of respiration rate-related features or parameters.

While respiration information extracted from biophysical signals (e.g., cardiac/biopotential signals, photoplethysmographic signals, and/or ballistographic signals) are only an approximation to the true respiration function and only carries partial information about respiration, it has been experimentally determined and validated through clinical studies that are described herein, that respiration rate-related features have significant clinical utility in the assessment of the presence or non-presence of cardiac disease, including in the estimation of the presence of elevated or abnormal left-ventricular end-diastolic pressure (LVEDP), which is an established indicator of the onset of left heart failure. This is notable since the clinical studies demonstrate the clinical utility of the analytical system, and the algorithms described herein can be used as a replacement for more complex direct or indirect measurement systems. True respiration is conventionally measured using a device that measures the air inflow and outflow rate to the lungs. An indirect method such as impedance pneumography, as an alternative to direct airflow measurement, requires complex hardware that provides an approximation of respiration by interrogating the expansion of the chest wall due to respiration.

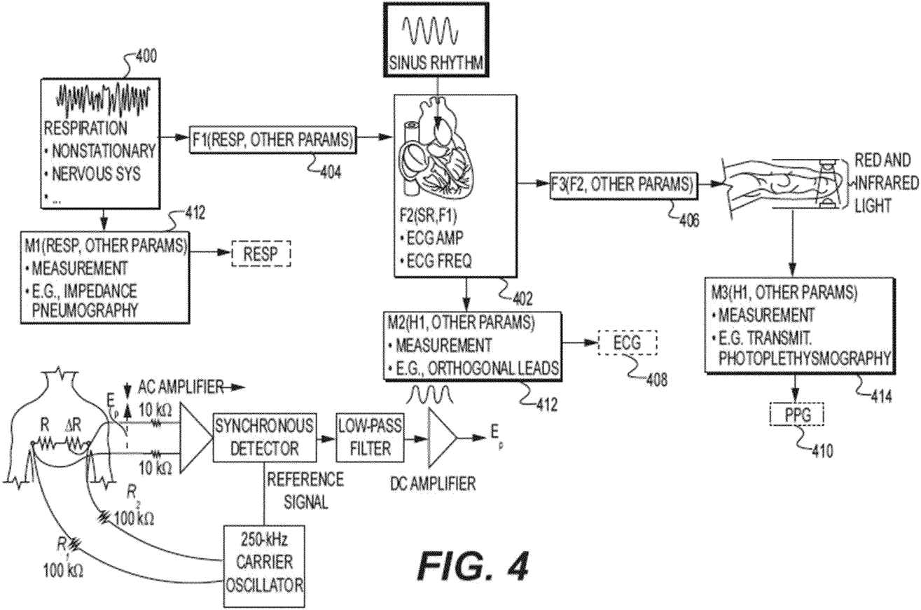

Indeed, as shown in FIG. 4, as the respiration effect reaches the heart, its modulating effect on the acquired biopotential/cardiac signals (e.g., 104b) becomes a secondary effect because it passes through other nonlinear transfer functions and will be diluted by noise and other physiological parameters. Similarly, by the time the respiration modulating effect appears in the PPG signals (e.g., 104a), it has gone through various functional blocks and has transformed nonlinearly. In FIG. 4, the respiration information, R, is shown to be diluted or modulated with heart noise F₂ (402) and other physiological parameters F₁ (404) and F₃ (406), each of which may be non-linear. In addition, it can be seen that the acquired biophysical-signal data 110 of the biophysical signal recorder system (shown as “ECG” 408 and “PPG” 410) may introduce additional non-linearity “M₂” (412) and “M₃” (414) to the signal of interest in which the estimated respiration rate-related information, R_{est,} can be modeled as Rₑₛₜ =M3-F3 F₂-F₁R (for a PPG signal) and as Rₑₛₜ = M₂F₂F₁R (for a cardiac signal).

Notably, even with such dilution, the indirect measurement of respiration information using the analytical system and algorithms disclosed herein is experimentally determined to have clinical utility in the assessment of the presence or non-presence of cardiac disease. Specifically, the selection of respiration rate-related features or parameters in an algorithm to estimate for the presence or non-presence of elevated or abnormal LVEDP is evidence of the power of the exemplary system in being able to use indirect observers (e.g., via measurements “ECG” and “PPG” signals) to make a clinically relevant estimation of the metrics of a physiological system of a patient. A direct observer would have less dilution in its acquired signal (e.g., Resp = MrR), but at a potential cost of additional or more complex hardware. It is noted that the various systems and methods described herein do not require that the observable measure functions “M₂” and “M₃” nor the transfer functions “F₁,” “F₂,” and “F₃” be solved.

### Respiration Rate-Related Features Computation Modules

FIGS. 5-9 each shows an example respiration rate-related feature computation module, for a total of five example modules, configured to determine values of respiration rate-rated features or parameters in accordance with an illustrative embodiment. In particular, the respiration-rate feature assessment module 500 of FIG. 5 determines features or parameters associated with respiration rate from an acquired photoplethysmographic and biopotential/cardiac signals. Module 600 of FIG. 6 determines features or parameters associated with heart rate variability. Module 700 of FIG. 7 determines features or parameters associated with relative entropy, which quantifies the complexity of physiological information between one or more input modulated signals associated with respiration and a baseline modulated signal. Module 800 of FIG. 8 determines features or parameters that quantify the cross-spectral agreement between a synthetic respiration waveform and one or more input modulated signals associated with respiration. Module 900 of FIG. 9 determines features or parameters that assess the maximum mean discrepancy among calculated distances determined between an estimated power of a synthetic respiration waveform and the estimated powers of one or more input modulated signals associated with respiration. Module 900 may encode the distance with respect to a probability distribution. The assessment module 103, more specifically the analytical engine or analyzer therein, may call on specific feature functions within any of these modules 500, 600, 700, 800, 900 in whole or in part as described below for a given clinical application.

### Example #1 - Respiration Rate Estimation

FIG. 5 illustrates, as the first of five example feature categories, an example respiration-rate feature assessment module 500 configured to determine output values of respiration rate-related features or parameters that characterize respiration rate properties of a patient within an acquired biophysical-signal data set. Module 500 is configured, in some embodiments, to estimate a plurality of respiration rates for one or more, or each, of an acquired set of biophysical signals (e.g., photoplethysmographic and cardiac signals) by extracting a plurality of modulated signals using different types of modulation operators. The plurality of modulated signals are used to estimate a corresponding number of respiration rates, which are then fused together to generate a distribution (e.g., histogram) of respiration rate estimates. Subsequently, one or more statistical and/or geometric characterizations of the distribution are extracted as a feature set or parameter set for a classifier (e.g., module 116). Such characterization of a distribution from multiple analyses can account for nonlinearities in the coupling between the human respiratory system and the cardiac system as manifested in the instant observed measurements in biophysical signals (e.g., cardiac and/or PPG signals), e.g., as described in relation to FIG. 4.

Table 1 shows an example set of four extracted statistical and/or geometric characterizations of distribution of respiration rate estimations, including mean, standard deviation, kurtosis, and skewness. In Table 1, the mean of the distribution of respiration rate estimations, “dRRMean,” has been experimentally determined to have significant utility in the assessment of the presence or non-presence of at least one cardiac disease, medical condition, or an indication of either such as the determination of presence or non-presence of elevated LVEDP.

It has also been observed through experimentation that the distribution of assessed respiration rate has significant utility in the assessment of the presence or non-presence of coronary artery disease. The list of the specific features determined to have significant utility in the assessment of the presence or non-presence of abnormal or elevated LVEDP is provided in Tables 7A-7C, and the presence or non-presence of significant CAD is provided in Table 8.

TABLE 1

| No. | Feature name | Feature Description |
|---|---|---|
| 1 | dRRMean* | Mean, standard deviation, skewness, or kurtosis of the distribution of fused respiration rate estimations fused from a plurality of respiration rate estimators. |
| 2 | dRRStd | |
| 3 | dRRSkew | |
| 4 | dRRKurt* * | |

FIG. 10 shows a detailed implementation of the respiration-rate feature assessment module 500 (shown as 500a) of FIG. 5 in accordance with an illustrative embodiment, which can be used wholly, or partially, to generate respiration rate-related features or parameters and its outputs to be used in machine-learned classifier to determine a metric associated with a physiological system of a patient under study. To determine the features of Table 1, Module 500a is configured, in some embodiments, to (i) precondition an inputted biophysical-signal data set, (ii) delineate the preconditioned signals for landmark detection, (iii) extract modulated signals from the biophysical signals, (iv) process the modulated signals, (v) segment each modulated signal into windows, (vi) extract respiration rate values, (vii) combine via a fusion operation the calculated respiration rate values for each given modulation signal, and (viii) generate one or more features and their corresponding values as the output of the module.

FIG. 10 shows a set of modulation modules 1002-1012 that performs operations (i) —(iv), a respiration rate estimation and fusion module 1018 that performs operations (v) — (vii), and a feature output generation module 1022 that performs operations (viii). The output of module 500a includes one or more of the statistical or geometric characterizations of the determined distribution of respiration rate estimates, including the mean, standard deviation, skewness, and kurtosis of that distribution.

In FIG. 10, Module 500a is shown to include two sets of six different types of modulation modules 1002a-1012a and 1002b-1012b each configured to perform operations (i) -(iv), the six-module types include amplitude modulation modules 1002a, 1002b, frequency modulation modules 1004a, 1004b, peak modulation modules 1006a, 1006b, continuous-wavelet-transform (CWT) amplitude modulation modules 1008a, 1008b, continuous-wavelet-transform (CWT) frequency modulation modules 1010a, 1010b, and enhanced modulation modules 1012a, 1012b. The modulation modules 1002a-1012a and 1002b-1012b are configured to receive two acquired biophysical-signal data sets shown in this example to include i) a first biophysical-signal data set (e.g., having been additionally pre-processed and shown as 110a') for a photoplethysmographic signal set and ii) a second biophysical-signal data set (e.g., having been additionally pre-processed and shown as 110b') for a cardiac signal set. Module 500a can provide, in this example, a total of 30 modulated signals (e.g., using five signals (i.e., cardiac signals “x,” “y,” “z” and PPG signals “U” and “L”) each being applied to the six modulation modules). The outputs of the 30 modulated signals are shown by 12 signal groups 1014a-10141.

(i) Precondition an Input Biophysical-Signal Data Set. To generate the respiration rate-related features and their outputs, a low-pass filter (not shown) may first be applied to the input biophysical signals 110a and 110b to remove frequencies that are above a given respiration range (e.g., using a low pass filter having a transition band at 0.8 Hz and 0.9 Hz) to generate the preconditioned signals 110a' and 110b'.

(ii) Delineate the preconditioned signals for landmark detection. Module 500a may then delineate (e.g., via modules 1002a-1012a and 1002b-1012b) the preconditioned signals of the preconditioned signals 110a' and 110b' using a landmark detection operation to identify peak values (Pₖ.ᵥ) and trough values (T_{r.v}) and their corresponding peak times (Pr.t) and trough times (Tr.t). The delineated landmarks may be used for certain subsequent analyses, e.g., amplitude, frequency, and PM modulations. An example of a peak detector is the Pan-Tompkins algorithm [12], which may be used to determine peaks, as well as troughs (e.g., by inverting the signals and performing the peak detections on the inverted signal). The incremental-merge segmentation (IMS) algorithm for PPG signals [13].

(iii) Extract modulation signals. Module 500a then uses the six different types of modulation modules 1002a-1012a and 1002b-1012b to extract a plurality of time-series signals (e.g., 30 modulation signals) as a set of modulated signals in which each modulated signal is dominated by respiration modulation. Plot 1024 shows an example AM modulated signal extracted from a patient’s PPG signal as a representative modulation signal of the 30 modulations signals. Other numbers and types of modulations may be used, for example, as described in [2].

Modules 1002a, 1002b, 1004a, 1004b, 1006a and 1006b perform the modulation using the delineated landmarks from step (ii).

Amplitude modulation (e.g., per modules 1002a, 1002b) uses, in some embodiments, the difference between the detected peak values (P_{k.v}) and the detected trough values (Tᵣ.ᵥ) in a given input signal per AM = Pₖ.ᵥ - Tᵣ (either in the cardiac signals or photoplethysmographic signals) to create the time-series signal.

Frequency modulation (e.g., per module 1004a, 1004b) uses, in some embodiments, the difference in time intervals between two peaks (i.e., FM = P_{k.t+1} - P_{k.t}) ( either in the cardiac signals or photoplethysmographic signals) to create the time-series signal.

Peak modulation (e.g., per modules 1006a, 1006b) uses, in some embodiments, the difference between peak values (PM = P_{k.v+1} - P_{k.v}) (either in the cardiac signals or photoplethysmographic signals) to create the time-series signal.

Continuous wavelet transform modulation. Module 500a applies a mother wavelet to the preconditioned signals 110a’, 110b' to generate AM modulation CWT signals (e.g., per modules 1008a, 1008b) and to generate the FM modulation CWT signals (e.g., per modules 1010a, and 1010b). The mother wavelet may be based on Morlet, Gaussian, Mexican Hat, Spline, Mayer wavelet, Wavelet kernels, etc. Modules 1008 and 1010 then each identifies, in some embodiments, the maximum intensity within the heart rate range (e.g., about 30 - 105 bpm). In some embodiments, for frequency modulation CWT, the frequency associated with the identified maximum is used to form the frequency CWT modulation signals, while for amplitude modulation CWT, the intensity associated with the maximum forms the amplitude CWT modulation signals. The respiration-rate feature assessment module 500a is configured, in some embodiments, to down-sample the biophysical signals (e.g., 250 Hz to 25 Hz) to improve the computation speed.

Enhanced modulation (e.g., per modules 1012a, 1012b) is performed, in some embodiments, using adaptive filters. The adaptive filter modules, in some embodiments, comprise a Weiner filter that can estimate an enhanced signal that lies between a strong and a weak signal with respect to signal-to-noise ratios, e.g., a ratio of the power of the fundamental frequency to that of noise and harmonics. In some embodiments, the strongest signal comprises the strongest fundamental frequency, having the largest absence of noise and harmonics, while the weakest signal comprises the opposite — the lowest presence of the fundamental frequency, having the most noise and/or harmonics. The enhanced modulation modules 1012a, 1012b are configured to denoise the strongest signal by using the weakest signal as a quantification of the “noise” signal.

(iv) Process the Modulation Signals. Following the extraction of the modulated signals, Module 500a (e.g., via modules 1002a-1012a and 1002b-1012b) is configured to resample the outputted modulated signals to fill in any missing values from the modulation extraction. The operation ensures there are no missing values in the data set. Modules 1002a-1012b and 1002b-1012b also include a filter to remove frequencies that are above and below the respiration range. In addition, modules 1002a-1012a and 1002b-1012b may apply a low-pass filter to remove frequencies that are above respiration range (e.g., with a transition band at about 0.8 and about 0.9 Hz) and apply a high-pass filter to remove sub-respiration frequencies (e.g., at transition band at about 0.02 and about 0.15 Hz).

(v) Segment Each Modulated Signal into Windows. Module 500a includes a respiration rate estimation and fusion module 1018 that receives the 30 outputs (in 1014a-10141) of modulation modules 1002-1012. Module 1018 then segments, in some embodiments, each of the 30 inputted modulated signals (in 1014a-10141) into a plurality of windows (e.g., having a window length of about 16 seconds with an overlap of about 8 seconds). To synchronize the timing between the types of biophysical signals, Module 1018 may identify the largest common intersections among the signals. The timing of the windows does not have to tally up to the total length of the signals.

(vi) Extract Respiration Rate Values. Module 1018 then executes a respiration extraction algorithm for all the windows of the modulated signals to generate a plurality of window respiration rate estimates. The respiration extraction algorithm, for each window, may compute a power spectral density (PSD) of the window, e.g., using autoregressive modeling (ARM) of orders 5 to 15, which is particularly well suited for very sparse data set. An example of an autoregressive PSD estimation operator is the “pburg” function in Matlab (manufactured by Mathworks, Natick, MA). The algorithm then identifies, in some embodiments, one or more peaks in the estimated PSD over the range of respiration, e.g., between about 6 and 20 breathes per minute (BPMᵣₑₛₚ). Plot 1026 shows an example respiration rate estimate over time derived from a given modulated signal.

(vii) Generate Distribution of Respiration Rate Estimates. Module 1018 then fuses the plurality of window respiration-rate estimates to generate a distribution (e.g., histogram) of respiration rate estimates. Plot 1028 shows an example distribution (e.g., histogram) of fused respiration rate estimates outputted by module 1018. In some embodiments, two or more distributions are generated, e.g., via multiple fusion operations #1, #2, and #3, and are aggregated wholly or partially together to generate a single distribution as the output of module 1018. In some embodiments, Module 1018 may send each calculated distribution to the feature output generation module 1022, which performs the aggregation of the calculated distributions.

Module 1018 may perform fusion operation #1 by identifying and aggregating the median respiration rate value identified for each window of the modulated signals (e.g., 30 modulated signals.) The aggregated respiration rate values are the outputs of Module 1018.

Module 1018 may perform fusion operation #2 using SNR weighting. For SNR weighting, Module 1018 may perform an assessed quality of the windows of the modulated signals (e.g., 30 modulated signals) and removes windows having a low assessed quality, e.g., (i) computing an SNR quality of each window and (ii) removing outlier windows having SNR beyond one median absolute deviation. The remaining windows may be combined (e.g., via median operator). In other embodiments, module 1018 may further (iii) create a weighted vector based on the SNR values and (iv) determine the fused output as weighted sums of the weighted vector and the window respiration rate estimates.

Module 1018 may perform fusion operation #3 by computing, for each window, an average ARM PSD for each of the modulated signals (e.g., 30 modulated signals) and aggregating identified peaks of the ARM PSD of the various windows of the modulated signals as the fused output. Other types of fusion may be used, for example, as described in [2].

(viii) Generate Features and Their Corresponding Values. Feature output generation module 1022 receives the output of the respiration rate estimation and fusion module 1018 in which the output includes one or more distributions of the respiration rate estimates. Module 1022 then computes the mean, standard deviation, skewness, and kurtosis of the distribution and outputs the values as the output(s) of module 500a.

### Example #2 - Heart Rate Variability Estimation

FIG. 6 illustrates, as the second of the five feature categories, an example heart rate variability feature assessment module 600 configured to determine output values of respiration rate-related features or parameters that characterize heart rate variability (HRV) properties of a patient within an acquired biophysical signal set. Module 600 is configured, in some embodiments, to estimate HRV using the FM modulation signals generated, for example, during the generation of the respiration rate estimations as described in relation to FIG. 10. The modulated signals may be segmented into windows and the HRV values extracted. The HRV values may be fused to generate an HRV distribution for each of the biophysical signal types, e.g., one for cardiac signals and another for PPG signals, to which statistical and/or geometric characterizations of the distributions can be extracted as a feature set for a classifier.

Table 2 shows an example set of four extracted statistical and/or geometric characterizations of the distribution of HRV estimations for each type of input biophysical signals, including mean, standard deviation, kurtosis, and skewness. In Table 2, the skewness of the distribution of HRV estimations, “dHRVStdPPG,” has been experimentally determined to have significant utility in the assessment of the presence or non-presence of at least one disease state, medical condition, or an indication of either such as the determination of presence or non-presence of elevated LVEDP. It has also been observed through experimentation that the distribution of assessed heart rate variability, “dHRVStdPPG” has significant utility in the assessment of the presence or non-presence of coronary artery disease. The list of the specific features determined to have significant utility in the assessment of the presence or non-presence of abnormal or elevated LVEDP is provided in Tables 7A-7C, and the presence or non-presence of significant CAD is provided in Table 8.

TABLE 2

| No. | Feature Name | Feature Description |
|---|---|---|
| 1 | dHRVMeanECG | Mean, standard deviation, skewness, or kurtosis of the distribution of assessed heart rate variability (HRV) estimations generated using cardiac signals. |
| 2 | dHRVStdECG | |
| 3 | dHRVSkewECG | |
| 4 | dHRVKurtECG | |
| 5 | dHRVMeanPPG | Mean, standard deviation, skewness, or kurtosis of the distribution of assessed heart rate variability (HRV) estimations generated using PPG signals. |
| 6 | dHRVStdPPG*^{,}** | |
| 7 | dHRVSkewPPG* | |
| 8 | dHRVKurtPPG | |

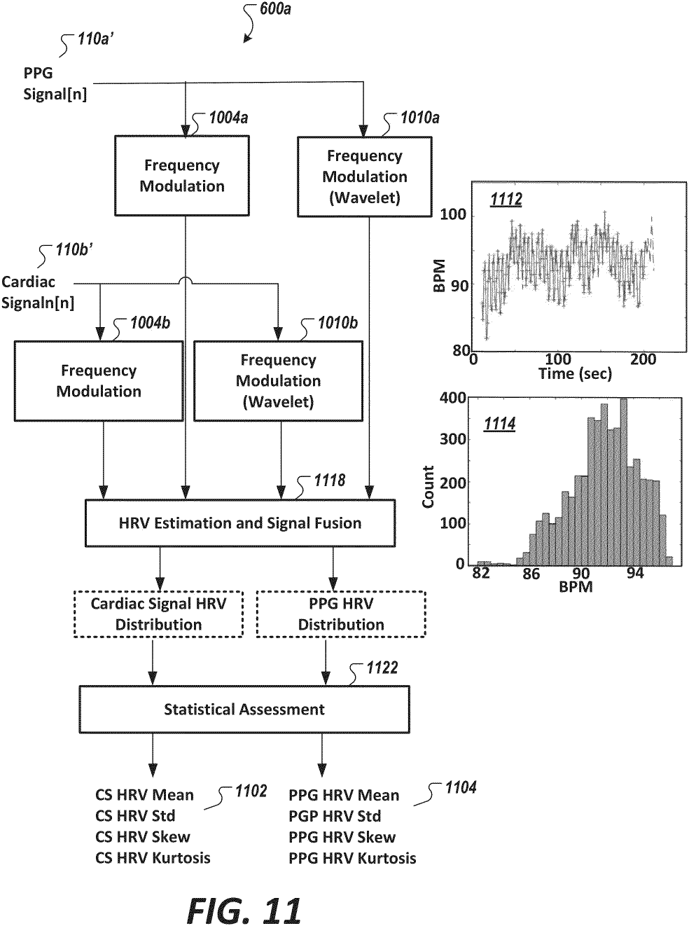

FIG. 11 shows a detailed implementation of the heart rate-variability (HRV) feature assessment module 600 (shown as 600a) of FIG. 6 in accordance with an illustrative embodiment, which can be used wholly, or partially, to generate HRV features or parameters and their outputs to be used in machine-learned classifier to determine a metric associated with a physiological system of a patient.

In FIG. 11, module 600a includes two types of modulation modules, namely the frequency modulation modules 1004a, 1004b and the continuous-wavelet-transform (CWT) frequency modulation modules 1010a, 1010b as described in relation to FIG. 10. The two modulation modules 1004 and 1010 are configured to receive the two pre-conditioned signal data sets 110a' and 110b' (e.g., comprising cardiac signals “x,” “y,” “z” and PPG signals “U” and “L”) to provide a total of 10 modulation signals. The two signal data sets 110a′ and 110b′ are evaluated to generate 4 features or parameters in this example per data set to provide a total of 8 features or parameters.

The modulation modules 1004a, 1004b, 1010a, and 1010b of module 600a may receive the pre-conditioned signals, delineate landmarks in the pre-conditioned signals, and extract the FM modulation signals and FM CWT modulation signals using the delineated landmarks, e.g., as described in relation to FIG. 10. In some embodiments, the same FM modulation signals and FM CWT modulation signals generated by module 500a may be used.

Module 600a includes an HRV estimation and signal fusion module 1118 that may operate in like manner to the respiration rate estimation and signal fusion module 1018. However, rather than processing the modulated signals to remove frequencies that are above the respiration range, the low-pass filter is configured to remove frequencies that are above the heartbeat range, and a high-pass filter is configured to remove sub-heartbeat frequencies. The outputted modulated signals may be resampled and segmented into windows, and heartbeat estimation values may be extracted (e.g., using an autoregressive PSD estimation operator) as described in relation to FIG. 10. Plot 1112 shows an example HRV signal generated from an FM modulated signal of a photoplethysmographic signal.

Module 1118 may then fuse the plurality of segmented HRV estimates to generate a distribution (e.g., histogram) of HRV estimates. In some embodiments, similar fusion operations and multiples of them, as described in relation to FIG. 10, may be performed. Module 1118 may generate a different distribution HRV estimate for each of the biophysical input types, e.g., one for cardiac signals (shown as 1104) and another for PPG signals (shown as 1102). Plot 1114 shows an example distribution (e.g., histogram) of fused HRV estimates outputted by module 1118.

Module 1122 then computes the mean, standard deviation, skewness, and kurtosis of the distribution of HRV estimates generated from PPG signals and cardiac signals and outputs the values as the output(s) of Module 600a.

### Example #3 - Relative Entropy Features

FIG. 7 illustrates, as the third of the five feature categories, an example relative entropy (RE) feature assessment module 700 configured to determine values of relative entropy features, as respiration rate-related features or parameters, that quantify the complexity of physiological information between an input modulated signal associated with respiration and a baseline modulated signal. A power spectral density determined from a given modulated signal is a complex amalgamation of various physiological and measurement effects, e.g., as described in relation to FIG. 4. The relative entropy features can provide a measure for this complexity as it imparts the influence of other physiological effects, which may be linked to a disease state, medical condition, or an indication of either. Module 700 may extract one or more statistical and/or geometric characterizations of the distribution of relative entropy for use in a classifier (e.g., module 116).

Table 3 shows an example set of 24 extracted statistical and/or geometric characterizations (e.g., mean, standard deviation, kurtosis, and skewness) of the distribution of assessed relative entropy estimations of a biophysical signal. The relative entropy estimations are each determined relative to a base entropy. In Table 3, seven features have been experimentally determined to have significant utility in the assessment of the presence or non-presence of at least one cardiac disease state, medical condition, or an indication of either such as the determination of presence or non-presence of elevated LVEDP. Of the 7 features, 4 features are directed to the mean of the distribution of relative entropy estimations derived from peak and amplitude modulations of both cardiac and photoplethysmographic signals; a 5^{th} is directed to a mean of a distribution derived from a frequency modulation of the cardiac signal; a 6^{th} is directed to a standard deviation of a distribution derived from an amplitude modulation of the photoplethysmographic signal; a 7^{th} is directed to the skewness of a distribution derived from a frequency modulation of the photoplethysmographic signal.

It has also been observed through experimentation that the distribution of assessed relative entropy estimations derived from peak, amplitude, and frequency modulations of cardiac and photoplethysmographic signals has significant utility in the assessment of the presence or non-presence of coronary artery disease. The list of the specific features determined to have significant utility in the assessment of the presence or non-presence of abnormal or elevated LVEDP is provided in Tables 7A-7C, and the presence or non-presence of significant CAD is provided in Table 8.

TABLE 3

| No. | Feature Name | Feature Description |
|---|---|---|
| 1 | dPkECGEntMean* | Mean, standard derivation, skewness, or kurtosis of the distribution of assessed relative entropy estimations of a |
| 2 | dPkECGEntStd | |
| 3 | dPkECGEntSkew | |
| 4 | dPkECGEntKurt** | modulated cardiac signal to a baseline modulated signal in which the cardiac signal is modulated by peak modulation. |
| 5 | dAmECGEntMean* | Mean, standard derivation, skewness, or kurtosis of the distribution of assessed relative entropy estimations of a modulated cardiac signal to a baseline modulated signal in which the cardiac signal is modulated by amplitude modulation. |
| 6 | dAmECGEntStd | |
| 7 | dAmECGEntSkew | |
| 8 | dAmECGEntKurt | |
| 9 | dFmECGEntMean* | Mean, standard derivation, skewness, or kurtosis of the distribution of assessed relative entropy estimations of a modulated cardiac signal to a baseline modulated signal in which the cardiac signal is modulated by frequency modulation. |
| 10 | dFmECGEntStd | |
| 11 | dFmECGEntSkew | |
| 12 | dFmECGEntKurt | |
| 13 | dPkPPGEntMean* | Mean, standard deviation, skewness, or kurtosis of the distribution of assessed relative entropy estimations of a modulated PPG signal to a baseline modulated signal in which the photoplethysmographic signal is modulated by peak modulation. |
| 14 | dPkPPGEntStd | |
| 15 | dPkPPGEntSkew | |
| 16 | dPkPPGEntKurt | |
| 17 | dAmPPGEntMean* | Mean, standard deviation, skewness, or kurtosis of the distribution of assessed relative entropy estimations of a modulated PPG signal to a baseline modulated signal in which the photoplethysmographic signal is modulated by amplitude modulation. |
| 18 | dAmPPGEnt Std *^{,}** | |
| 19 | dAmPPGEntSkew | |
| 20 | dAmPPGEntKurt | |
| 21 | dFmPPGEntMean | Mean, standard deviation, skewness, or kurtosis of the distribution of assessed relative entropy estimations of a modulated PPG signal to a baseline modulated signal in which the photoplethysmographic signal is modulated by frequency modulation. |
| 22 | dFmPPGEntStd | |
| 23 | dFmPPGEntSkew* | |
| 24 | dFmPPGEntKurt** | |

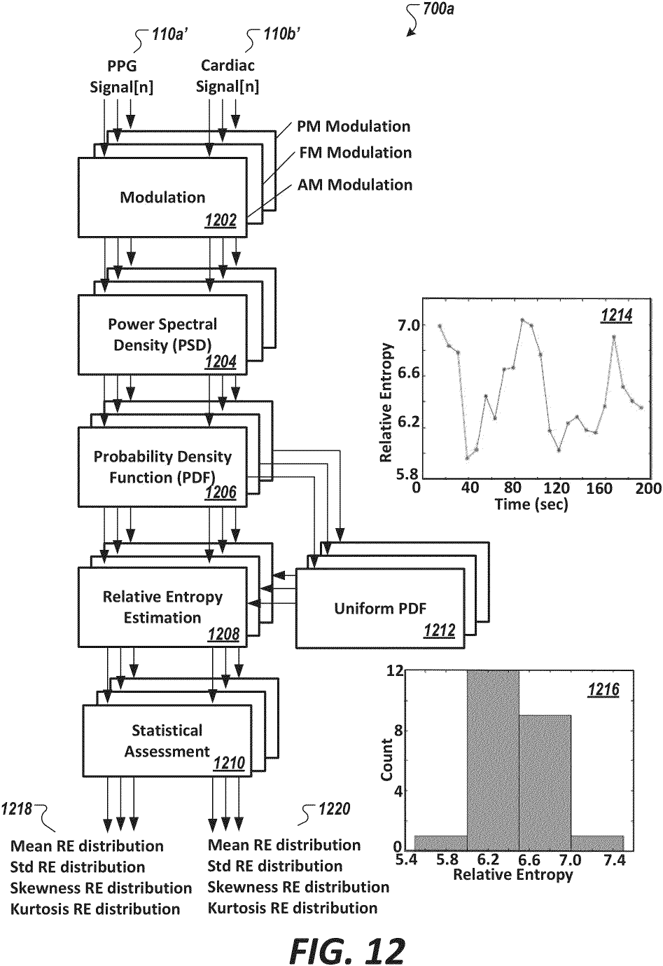

FIG. 12 shows a detailed implementation of the relative entropy feature assessment module 700 (shown as 700a) of FIG. 7 in accordance with an illustrative embodiment, which can be used wholly, or partially, to generate respiration rate-related features or parameters and its outputs to be used in a classifier to determine a metric associated with a physiological system of a patient. In FIG. 12, Module 700a includes three types of modulation modules (shown as modules 1202), namely the amplitude modulation modules 1002a, 1002b, frequency modulation modules 1004a, 1004b, and peak modulation modules 1006a, 1006b as described in relation to FIG. 10. The three modulation modules 1202 are configured to receive the two pre-conditioned signal data sets 110a' and 110b' (i.e., cardiac signals “x,” “y,” “z” and photoplethysmographic signals “U” and “L”) to generate a total of 15 modulated signals. The two signal data sets 110a' and 110b' are evaluated for three different modulation types, which generate 4 features or parameters in this example per data set and modulation type to provide a total of 24 features or parameters.

The modulation modules 1202 may receive the pre-conditioned signal data sets, delineate landmarks in the pre-conditioned signal data sets, and extract the AM, FM, and peak modulated signals from the pre-conditioned signal data sets as described in relation to FIG. 10. In some embodiments, the same AM, FM, and peak modulated signals generated by Module 500a may be used.

Module 700a further includes power spectral density assessment modules 1204 (shown as “Power Spectral Density (PSD)” modules 1204), probability density function assessment modules 1206 (shown as “PSD to PDF Conversion” modules 1206), relative entropy estimation modules 1208, and statistical assessment modules 1210.

Power Spectral Density assessment modules 1204 are each configured to receive the plurality of modulated signals (e.g., 15 modulated signals) and perform power spectral analysis (PSA) of each of the modulated signals to generate a plurality of PSD signals. Module 1204 may analyze the signal energy (e.g., power) of each modulated signal in the frequency domain by decomposing the modulated signal as a time-series signal into its frequency components. In this example, Module 1204 is configured to segment each of the modulation signals into windows, and a PSD window signal is generated for each segment of a modulation signal.

Probability density function assessment modules 1206 are each configured, in some embodiments, to receive the plurality of PSD window signals for a given modulation signal from a corresponding module 1204 and convert each of the received PSD window signals for that modulated signal to a probability density function (PDF) window signal. To do so, each of Modules 1206 may calculate the area under the power spectral curve of a given PSD window signal and then normalizing that PSD window signal with its calculated area under the power spectral curve.

Relative entropy estimation modules 1208 are each configured to receive the plurality of normalized PSD window signals and define a uniform probability distribution (another term for PDF) for the frequency range of each of the normalized PSD window signals (shown as “Uniform PDF” modules 1212). Other probability distributions may be used, e.g., normal, etc. Modules 1208 then calculate values for a plurality of relative entropy, RE_{window}, defined between the plurality of PDF window signals, ppsa(x), and its uniform probability distribution, puny(x), per Equation 1:

$$R {E}_{w i n d o w} = \int {p}_{u n i f} \left(f\right) log \left(\frac{{p}_{u n i f} \left(x\right)}{{p}_{p s d} \left(x\right)}\right)$$

In Equation 1, p(.) is the PSD window signal for a given modulation signal, x, (e.g., AM, FM, or PM modulation signals). Plot 1214 shows an example relative entropy signal generated from an FM modulation signal of a cardiac signal.

Modules 1208 then aggregates the calculated relative entropy values for a given modulated signal (e.g., 15 modulated signals) to generate a distribution of relative entropy estimation for that modulated signal. Plot 1216 shows an example distribution of relative entropy estimations for the relative entropy signal of plot 1216.

Statistical assessment modules 1210 are each configured, in some embodiments, to receive the distribution of relative entropy estimations for each of the modulated signals and to compute the mean, standard deviation, skewness, and kurtosis of the distribution for the given modulated signal as the output(s) (1218 and 1220) of module 700a for each of the biophysical signal types.

### Example #4 - Maximum Mean Discrepancy (MMD) Distance Features

FIG. 8 illustrates, as the fourth of the five feature assessment categories, an example maximum mean discrepancy distance feature assessment module 800 configured to determine values of maximum mean discrepancy distance features, as respiration rate-related features or parameters, that quantify a difference between the signal energy of given modulated signal and the signal energy of respiration information (effect size) on average. Module 800 constructs, in some embodiments, a proxy respiration signal (also referred to as an estimated respiration waveform) from an estimated respiration rate as determined from Module 500. The MMD distance estimation may be a calculated difference between a computed power spectral density of the proxy respiration signal and a computed power spectral density of each of the plurality of modulated signals. Module 800 may then compute/extract one or more features that are statistical and/or geometric characterizations of the distribution of MMD distance estimations for use in a classifier (e.g., module 116).

Table 4 shows an example set of 24 extracted statistical and/or geometric characterizations (e.g., mean, standard deviation, kurtosis, and skewness) of the distribution of assessed maximum mean discrepancy distance estimations of a biophysical signal. The maximum mean discrepancy distance estimations are each determined as a power density function of a given modulation signal of an input biophysical signal relative to a power density function of a proxy respiration waveform. In Table 4, four features have been experimentally determined to have significant utility in the assessment of the presence or non-presence of at least one cardiac disease state, medical condition, or an indication of either such as the determination of presence or non-presence of elevated LVEDP. The 4 features include skewness and kurtosis (2 features) of the distribution of maximum mean discrepancy distance estimations derived from amplitude modulation of a cardiac signal and a mean and kurtosis (another 2 features) of the distribution of maximum mean discrepancy distance estimations derived from amplitude modulation of a PPG signal.

It has also been observed through experimentation that the distribution of assessed maximum mean discrepancy distance derived from peak, amplitude, and frequency modulations of cardiac and photoplethysmographic signals have significant utility in the assessment of the presence or non-presence of coronary artery disease. The list of the specific features determined to have significant utility in the assessment of the presence or non-presence of abnormal or elevated LVEDP is provided in Tables 7A-7C, and the presence or non-presence of significant CAD is provided in Table 8.

TABLE 4

| No. | Feature Name | Feature Description |
|---|---|---|
| 1 | dPkECGMMDMean | Mean, standard deviation, skewness, or kurtosis of the distribution of assessed maximum mean discrepancy distance estimations between PDF of a cardiac signal and a power density function of a proxy respiration rate in which the cardiac signal is modulated by peak modulation. |
| 2 | dPkECGMMDStd | |
| 3 | dPkECGMMDSkew | |
| 4 | dPkECGMMDKurt | |
| 5 | dAmECGMMDMean** | Mean, standard deviation, skewness, or kurtosis of the distribution of assessed maximum mean discrepancy distance estimations between PDF of a cardiac signal and a power density function of a proxy respiration rate in which the cardiac signal is modulated by amplitude modulation. |
| 6 | dAmECGMMDStd** | |
| 7 | dAmECGMMD Skew* | |
| 8 | dAmECGMMDKurt* | |
| 9 | dFmECGMMDMean** | Mean, standard deviation, skewness, or kurtosis of the distribution of assessed maximum mean discrepancy distance estimations between PDF of a cardiac signal and a power density function of a proxy respiration rate in which the cardiac signal is modulated by frequency modulation. |
| 10 | dFmECGMMDStd | |
| 11 | dFmECGMMDSkew | |
| 12 | dFmECGMMDKurt | |
| 13 | dPkPPGMMDMean | Mean, standard deviation, skewness, or kurtosis of the distribution of assessed maximum mean discrepancy distance estimations between PDF of a PPG signal and a power density function of a proxy respiration rate in which the PPG signal is modulated by peak modulation. |
| 14 | dPkPPGMMDStd | |
| 15 | dPkPPGMMD Skew** | |
| 16 | dPkPPGMMDKurt | |
| 17 | dAmPPGMMDMean* | Mean, standard deviation, skewness, or kurtosis of the distribution of assessed maximum mean discrepancy distance estimations between PDF of a PPG signal and a power density function of a proxy respiration rate in which the PPG signal is modulated by amplitude modulation. |
| 18 | dAmPPGMMDStd** | |
| 19 | dAmPPGMMDSkew | |
| 20 | dAmPPGMMDKurt* | |
| 21 | dFmPPGMMDMean** | Mean, standard deviation, skewness, or kurtosis of the distribution of assessed maximum mean discrepancy distance estimations between PDF of a PPG signal and a power density function of a proxy respiration rate in which the PPG signal is modulated by frequency modulation. |
| 22 | dFmPPGMMDStd | |
| 23 | dFmPPGMMDSkew** | |
| 24 | dFmPPGMMDKurt | |

FIG. 13A shows a detailed implementation of the maximum mean discrepancy distance feature assessment module 800 (shown as 800a) of FIG. 8 in accordance with an illustrative embodiment, which can be used wholly, or partially, to generate respiration rate-related features and its outputs to be used in a classifier to determine a metric associated with a physiological system of a patient. In FIG. 13A, Module 800a includes three types of modulation modules (shown as modules 1202 as described in relation to FIGS. 10 and 12) that receive the pre-processed five signals in the first and second biophysical-signal data sets 110a' and 110b' for a photoplethysmographic signal set and a cardiac signal set to provide a total of 15 modulation signals (e.g., cardiac signals “x,” “y,” “z” and PPG signals “U” and “L”). Module 800a further includes, in some embodiments, and as shown in FIG. 13A, power spectral density assessment modules 1204 and probability density function assessment modules 1206 to generate a plurality of power-density-function window signals, from the output of modules 1202, corresponding to segments of the plurality of modulated signals of the biophysical-signal data set, as described in relation to FIGS. 10 and 12.

Module 800a further includes a waveform generation module 1302 (to construct a proxy respiration signal) and a corresponding set of modulation modules 1202', power spectral density assessment modules 1204', and probability density function assessment modules 1206' to generate a plurality of power-density-function window signals of the proxy respiration signal, e.g., in a manner like that described in relation to modules 1202, 1204, and 1206.

Module 800a also includes maximum mean discrepancy (MMD) distance feature assessment modules 1304 configured to calculate MMD distance estimations, MMD, per Equation 2:

$$\begin{matrix}M M D = \frac{1}{{N}^{2}} {\sum }_{i , j} κ \left({x}_{m o d , i} , {x}_{m o d , j}\right) + \frac{1}{{N}^{2}} {\sum }_{i , j} κ \left({x}_{p r o x i , j} , {x}_{p r o x , j}\right) - \\ \frac{2}{{N}^{2}} {\sum }_{i , j} κ \left({x}_{m o d , i} , {x}_{p r o x , j}\right)\end{matrix}$$

In Equation 2, random samples (x_{mod}, _{Xprox}) are drawn for the frequency range of the PSD from each of the PDF window signals, and k(x, y) is a Gaussian kernel defined by Equation 3:

$$κ \left(x , y\right) = \frac{1}{\sqrt{2 π σ}} exp \left(- \frac{{\left|x - y\right|}^{2}}{2 {σ}^{2}}\right)$$

In Equation 3, σ is the standard deviation set to be equal to the minimum of that between x, y. Plot 1308 shows an example MMD distance estimation signal generated from an FM modulation signal of a cardiac signal.

Modules 1304 then aggregates the calculated maximum mean discrepancy distance values for a given modulation signal to generate a distribution of maximum mean discrepancy distance estimation for that modulated signal. Plot 1310 shows an example distribution of maximum-mean discrepancy distance estimations for the maximum mean discrepancy distance estimation signal of plot 1308.

Statistical assessment modules 1306 are each configured, in some embodiments, to receive the distribution of maximum mean discrepancy distance estimation for a given modulated signal and to compute the mean, standard deviation, skewness, and kurtosis of the distribution for the given modulated signal as the output(s) (shown as 1312 and 1314) of Module 800a.

Respiration Waveform Generator. FIG. 13B shows a detailed implementation of the waveform generation module 1302 of FIG. 13A in accordance with an illustrative embodiment. Module 1302 is configured to estimate a proxy respiration waveform from the estimated respiration rate, e.g., derived from biophysical-signal data sets as described in relation to FIG. 10. Module 1302 is configured, in some embodiments, to generate a proxy respiration waveform having the functional form of Equation 4:

$$S \left(t\right) = A \left(t\right) s i n \left(θ \left(t\right)\right)$$

In Equation 4, S(t) is the proxy respiration waveform, A(t) is an amplitude modulation function, and θ(t) is a phase function. Module 1302 includes a respiration rate estimation module 1018' configured to receive a respiration rate signal obtained from a respiration rate fusion operation (e.g., performed by module 1018) as described in relation to FIG. 10. Module 1018' computes the phase function θ(t) as an integration of the received respiration rate signals. Plot 1318 shows an example respiration rate signal (resampled) obtained from module 1018' (shown in Hz).

Module 1302 further includes a phase function module 1312, an amplitude function module 1314, and a proxy waveform generation module 1314. Phase function module 1312 is configured to determine phase function θ(t) by (i) using a respiration rate fused output (e.g., using any of the signal fusion methods described herein, e.g., median respiration rate fusion, SNR weighting, or power spectral density averaging) as a respiration rate signal; (ii) converting respiration rate signal from breaths per minute to Hz; (iii) sampling the converted respiration rate (in Hz) signal with the sampling frequency of a modulation signal; and (iv) integrating the time series according to θ(t) = 2 π J RR(t)dt.

Module 1314 is configured to compute the amplitude modulation function A(t) by identifying a modulation signal having the greatest number of windows with the largest SNR values; (ii) determining the envelope signal, envelope, of the identified modulation signal, e.g., using a Hilbert transform; and (iii) determining A(t) = 1 + envelope. Plot 1320 shows an example envelope signal generated from the example respiration rate signal of plot 1318.

Module 1316 is configured to generate the proxy waveform 1315 per Equation 4. Plot 1322 shows a proxy waveform signal 1315 generated from the example respiration rate signal of plot 1318.

### Example #5 - Coherence Features

FIG. 9 illustrates, as the fifth of five feature assessment categories, an example coherence feature assessment module 900 configured to determine coherence features, as respiration rate-related features or parameters, that quantify the cross-spectral similarity between a proxy respiration signal and each modulation input signal. Coherence can provide a measure showing the degree that a proxy waveform and the modulation signal groups are linearly related in the frequency domain. Module 900 may extract one or more statistical and/or geometric characterizations of the distribution of coherence estimations for a classifier (e.g., to be used in Module 116).

Table 5 shows an example set of 24 extracted statistical and/or geometric characterizations (e.g., mean, standard deviation, kurtosis, and skewness) of the distribution of assessed coherence estimations of a biophysical signal. The coherence estimations are each determined as power spectral densities and cross power spectral densities of the proxy waveform and modulated signal groups. In Table 5, three features have been experimentally determined to have significant utility in the assessment of the presence or non-presence of at least one cardiac disease state, medical condition, or an indication of either such as the determination of presence or non-presence of elevated LVEDP. The 3 features include the mean (2 features) of the distribution of coherence estimations derived from a peak and frequency modulation of a PPG signal a standard deviation (1 feature) of the distribution of coherence estimations derived from amplitude modulation of a cardiac signal.

It has also been observed through experimentation that the distribution of assessed coherence estimations derived from peak, amplitude, and frequency modulations of cardiac and photoplethysmographic signals has significant utility in the assessment of the presence or non-presence of coronary artery disease. The list of the specific features determined to have significant utility in the assessment of the presence or non-presence of abnormal or elevated LVEDP is provided in Tables 7A-7C, and the presence or non-presence of significant CAD is provided in Table 8.

TABLE 5

| No. | Feature Name | Feature Description |
|---|---|---|
| 1 | dPkECGCXYMean | Mean, standard deviation, skewness, or kurtosis of the distribution of assessed coherence estimations between a modulated cardiac signal and an estimated respiration waveform in which the cardiac signal is modulated by peak modulation. |
| 2 | dPkECGCXYStd | |
| 3 | dPkECGCXYSkew** | |
| 4 | dPkECGCXYKurt | |
| 5 | dAmECGCXYMean** | Mean, standard deviation, skewness, or kurtosis of the distribution of assessed coherence estimations between a modulated cardiac signal and an estimated respiration waveform in which the cardiac signal is modulated by amplitude modulation. |
| 6 | dAmECGCXYStd* | |
| 7 | dAmECGCXYSkew | |
| 8 | dAmECGCXYKurt | |
| 9 | dFmECGCXYMean | Mean, standard deviation, skewness, or kurtosis of the distribution of assessed coherence estimations between a modulated cardiac signal and an estimated respiration waveform in which the cardiac signal is modulated by frequency modulation. |
| 10 | dFmECGCXYStd | |
| 11 | dFmECGCXYSkew | |
| 12 | dFmECGCXYKurt* * | |
| 13 | dPkPPGCXYMean*^{,}* * | Mean, standard deviation, skewness, or kurtosis of the distribution of assessed coherence estimations between a modulated PPG signal and an estimated respiration waveform where the PPG signal is modulated by peak modulation. |
| 14 | dPkPPGCXYStd* * | |
| 15 | dPkPPGCXYSkew | |
| 16 | dPkPPGCXYKurt | |
| 17 | dAmPPGCXYMean | Mean, standard deviation, skewness, or kurtosis of the distribution of assessed coherence estimations between a modulated PPG signal and an estimated respiration waveform where the PPG signal is modulated by amplitude modulation. |
| 18 | dAmPPGCXYStd | |
| 19 | dAmPPGCXYSkew | |
| 20 | dAmPPGCXYKurt | |
| 21 | dFmPPGCXYMean* | Mean, standard deviation, skewness, or kurtosis of the distribution of assessed coherence estimations between a modulated PPG signal and an estimated respiration waveform where the PPG signal is modulated by frequency modulation. |
| 22 | dFmPPGCXYStd | |
| 23 | dFmPPGCXYSkew | |
| 24 | dFmPPGCXYKurt | |

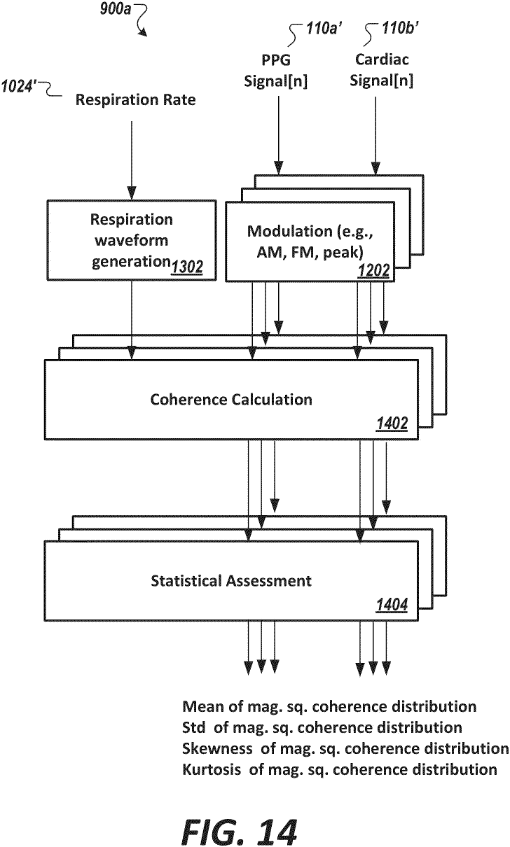

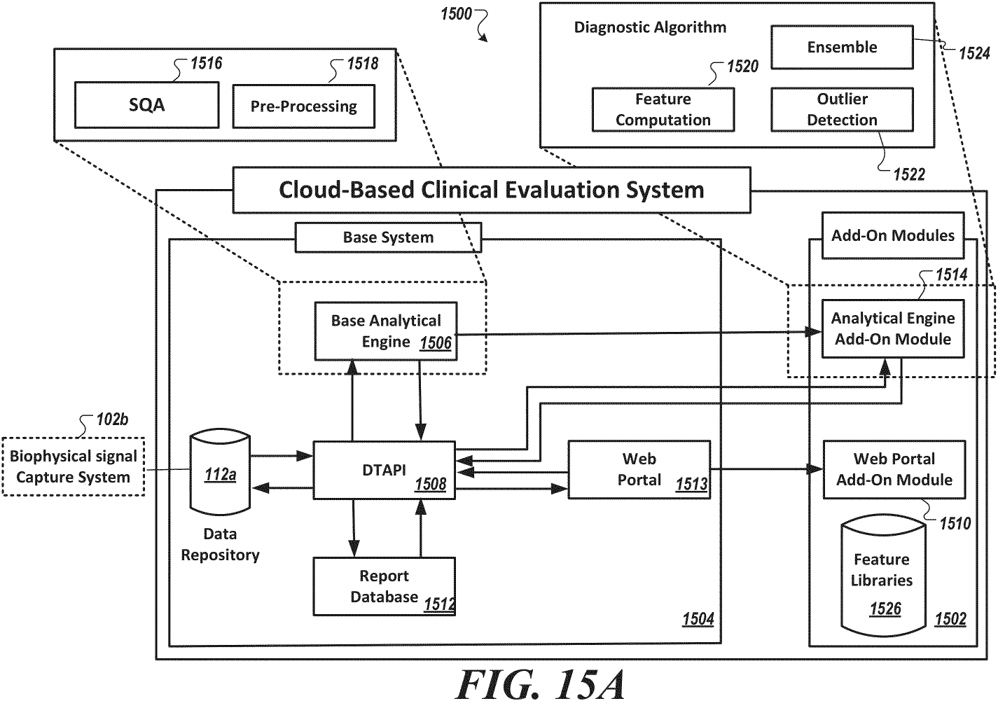

FIG. 14 shows a detailed implementation of the coherence feature assessment module 900 (shown as 900a) of FIG. 9 in accordance with an illustrative embodiment, which can be used wholly or partially, to generate respiration rate-related features or parameters and its outputs to be used in a classifier to determine a metric associated with a physiological system of a patient. In FIG. 14, module 900a includes three types of modulation modules (shown as modules 1202 as described in relation to FIGS. 10 and 12) and a respiration waveform generation module (shown as module 1302 as described in relation to FIG. 13).

Module 900a further includes a set of coherence calculation modules 1402 and statistical assessment modules 1404. Modules 1402 are each configured to calculate coherence per Equation 5:

$${C}_{x y} \left(f\right) = \frac{{\left|{P}_{x y} \left(f\right)\right|}^{2}}{{P}_{x x} \left(f\right) {P}_{y y} \left(f\right)}$$

In Equation 5, coherence is determined as a magnitude-squared coherence estimate C_{xy}(f) and is provided as a function of frequency with values between “0” and “1”. The magnitude-squared coherence estimate C_{xy}(f) indicates the degree to which a modulated signal x corresponds to the modulated respiration waveform signal y at each frequency for a given set of frequencies. The magnitude-squared coherence is a function of the power spectral densities, Pₓₓ(f) and Pyy(f), and the cross power spectral density, Pxy(f).

Modules 1402 then aggregates the calculated magnitude square coherence values for a given modulated signal to generate a distribution of magnitude sequence coherence estimation for that modulated signal. Statistical assessment modules 1404 are each configured, in some embodiments, to receive the distribution of magnitude sequence coherence estimation for a given modulation signal and to compute the mean, standard deviation, skewness, and kurtosis of the distribution for the given modulation signal as the output(s) of module 900a.

### Experimental Results and Examples

Several development studies have been conducted to develop feature sets, and in turn, algorithms that can be used to estimate the presence or non-presence, severity, or localization of diseases, medical conditions, or an indication of either. In one study, algorithms were developed for the non-invasive assessment of abnormal or elevated LVEDP. As noted above, abnormal or elevated LVEDP is an indicator of heart failure in its various forms. In another development study, algorithms and features were developed for the non-invasive assessment of coronary artery disease.

As part of these two development studies, clinical data were collected from adult human patients using a biophysical signal capture system and according to protocols described in relation to FIG. 2. The subjects underwent cardiac catheterization (the current “gold standard” tests for CAD and abnormal LVEDP evaluation) following the signal acquisition, and the catheterization results were evaluated for CAD labels and elevated LVEDP values. The collected data were stratified into separate cohorts: one for feature/algorithm development and the other for their validation.

Within the feature development phases, features were developed, including the respiration rate-related features, to extract characteristics in an analytical framework from biopotential signals (as an example of the cardiac signals discussed herein) and photo-absorption signals (as examples of the hemodynamic or photoplethysmographic discussed herein) that are intended to represent properties of the cardiovascular system. Corresponding classifiers were also developed using classifier models, linear models (e.g., Elastic Net), decision tree models (XGB Classifier, random forest models, etc.), support vector machine models, and neural network models to non-invasively estimate the presence of an elevated or abnormal LVEDP. Univariate feature selection assessments and cross-validation operations were performed to identify features for use in machine learning models (e.g., classifiers) for the specific disease indication of interest. Further description of the machine learning training and assessment are described in a U.S. provisional patent application concurrently filed herewith entitled “Method and System to Non-Invasively Assess Elevated Left Ventricular End-Diastolic Pressure” having attorney docket no. 10321-048pv1, which is hereby incorporated by reference herein in its entirety.

The univariate feature selection assessments evaluated many scenarios, each defined by a negative and a positive dataset pair using a t-test, mutual information, and AUC-ROC evaluation. The t-test is a statistical test that can determine if there is a difference between two sample means from two populations with unknown variances. Here, the t-tests were conducted against a null hypothesis that there is no difference between the means of the feature in these groups, e.g., normal LVEDP vs. elevated (for LVEDP algorithm development); CAD- vs. CAD+ (for CAD algorithm development). A small p-value (e.g., ≤ 0.05) indicates strong evidence against the null hypothesis.

Mutual information (MI) operations were conducted to assess the dependence of elevated or abnormal LVEDP or significant coronary artery disease on certain features. An MI score greater than one indicates a higher dependency between the variables being evaluated. MI scores less than one indicate a lower dependency of such variables, and an MI score of zero indicates no such dependency.

A receiver operating characteristic curve, or ROC curve, illustrates the diagnostic ability of a binary classifier system as its discrimination threshold is varied. The ROC curve may be created by plotting the true positive rate (TPR) against the false positive rate (FPR) at various threshold settings. AUC-ROC quantifies the area under a receiver operating characteristic (ROC) curve - the larger this area, the more diagnostically useful the model is. The ROC, and AUC-ROC, value is considered statistically significant when the bottom end of the 95% confidence interval is greater than 0.50.

Table 6 shows an example list of the negative and a positive dataset pair used in the univariate feature selection assessments. Specifically, Table 6 shows positive datasets being defined as having an LVEDP measurement greater than 20 mmHg or 25 mmHg, and negative datasets were defined as having an LVEDP measurement less than 12 mmHg or belonging to a subject group determined to have normal LVEDP readings.

TABLE 6

| | |
|---|---|
| Negative Dataset | Positive Dataset |
| ≤ 12 (mmHg) | ≥20 (mmHg) |
| ≤ 12 (mmHg) | ≥25 (mmHg) |
| Normal LVEDP | ≥20 (mmHg) |
| Normal LVEDP | ≥25 (mmHg) |

Tables 7A, 7B, and 7C each shows a list of respiration rate-related features having been determined to have utility in estimating the presence and non-presence of elevated LVEDP in an algorithm executing in a clinical evaluation system. The features of Tables 7A, 7B, and 7C and corresponding classifiers have been validated to have clinical performance comparable to the gold standard invasive method to measure elevated LVEDP.

TABLE 7A

Univariate Feature Assessment: LVEDP <= 12 (N=246) vs >=20 (N=209)

| Feature Name | t-test p-value | AUC (bottom of 95% CI) | MI |
|---|---|---|---|
| dAmECGEntMean | 0.0008 | 0.5562 | 1.2476 |
| dAmECGCXYStd | 0.0203 | n/s | n/s |
| dAmECGMMDKurt | 0.0263 | n/s | n/s |
| dAmECGMMDSkew | 0.0467 | n/s | n/s |
| dAmPPGEntMean | 0.0029 | 0.5212 | n/s |
| dAmPPGMMDKurt | 0.0098 | 0.5149 | n/s |
| dFmECGEntMean | 0.0036 | 0.5422 | 1.3541 |
| dFmPPGCXYMean | 0.0227 | n/s | n/s |
| dFmPPGEntSkew | 0.0346 | n/s | n/s |
| dHRVSkewPPG | 0.0121 | 0.5283 | 1.3422 |
| dHRVStdPPG | 0.0244 | n/s | n/s |
| dRRMean | 0.0192 | n/s | n/s |

TABLE 7B

Univariate Feature Assessment: LVEDP <= 12 (N=246) vs >=25 (N=78)

| Feature Name | t-test p-value | AUC (bottom of 95% CI) | MI |
|---|---|---|---|
| dPkPPGEntMean | 0.0013 | 0.5368 | n/s |
| dAmPPGEntStd | 0.0079 | n/s | 1.1745 |
| dPkECGEntMean | 0.0006 | 0.5999 | 1.4131 |
| dPkPPGEntStd | n/s | 0.5864 | 1.2446 |
| dAmPPGMMDMean | n/s | 0.5050 | 1.3631 |

TABLE 7C

Univariate Feature Assessment: Normal LVEDP group vs >=20 (N=78)

| Feature Name | t-test p-value | AUC (bottom of 95% CI) | MI |
|---|---|---|---|
| dPkPPGCXYMean | 0.0002 | n/s | 1.2713 |

Table 8 shows a list of respiration rate-related features having been determined to have utility in estimating the presence and non-presence of significant CAD in an algorithm executing in a clinical evaluation system. The features of Table 8 and corresponding classifiers have been validated to have clinical performance comparable to the gold standard invasive method to measure CAD.

TABLE 8

| Feature Name | t-test p-value | AUC | MI |
|---|---|---|---|
| dAmECGCXYMean | 0.0492 | 0.5019 | 1.2260 |
| dAmECGMMDMean | 0.0134 | n/s | n/s |
| dAmECGMMDStd | 0.0313 | 0.5032 | n/s |
| dAmPPGMMDStd | 0.0069 | 0.5061 | 1.0849 |
| dFmECGCXYKurt | n/s | n/s | 1.1344 |
| dFmECGMMDMean | n/s | n/s | 1.1801 |
| dFmPPGCXYMean | n/s | 0.5061 | 1.0133 |
| dFmPPGMMDMean | 0.0324 | 0.5084 | 1.0730 |
| dFmPPGMMDSkew | 0.0270 | n/s | n/s |
| dHRVStdPPG | 0.0263 | 0.5077 | n/s |
| dPkECGCXYSkew | 0.0231 | 0.5024 | n/s |
| dPkECGEntKurt | 0.0014 | 0.5331 | n/s |
| dPkECGMMDSkew | 0.0448 | n/s | n/s |
| dPkPPGCXYStd | 0.0470 | n/s | n/s |
| dPkPPGEntKurt | n/s | n/s | 1.0583 |
| dRRKurt | n/s | n/s | 1.3697 |

FA scenario = significant CAD (e.g., defined as > 70% blockage and/or FFR < 0.8) (N = 464; 232 CAD positives and 232 CAD negatives (½ single and (½ multi-vessel disease) (½ are males and ½ are females)

The determination that certain respiration rate-related features have clinical utility in estimating the presence and non-presence of elevated LVEDP or the presence and non-presence of significant CAD provides a basis for the use of these respiration rate-related features or parameters, as well as other features described herein, in estimating for the presence or non-presence and/or severity and/or localization of other diseases, medical condition, or an indication of either particularly, though not limited to, heart disease or conditions described herein.

The experimental results further indicate that intermediary data or parameters of respiration rate-related features, such as the synthesized respiration waveform, also have clinical utility in diagnostics as well as treatment, controls, monitoring, and tracking applications.

### Example Clinical Evaluation System

FIG. 15A shows an example clinical evaluation system 1500 (also referred to as a clinical and diagnostic system) that implements the modules of FIG. 1 to non-invasively compute respiration the rate-related features or parameters, along with other features or parameters, to generate, via a classifier (e.g., machine-learned classifier), one or more metrics associated with the physiological state of a patient or subject according to an embodiment. Indeed, the feature modules (e.g., of FIGS. 1, 5-14) can be generally viewed as a part of a system (e.g., the clinical evaluation system 1500) in which any number and/or types of features may be utilized for a disease state, medical condition, an indication of either, or combination thereof that is of interest, e.g., with different embodiments having different configurations of feature modules. This is additionally illustrated in FIG. 15A, where the clinical evaluation system 1500 is of a modular design in which disease-specific add-on modules 1502 (e.g., to assess for elevated LVEDP or mPAP, CAD, PH/PAH, abnormal LVEF, HFpEF, and others described herein) are capable of being integrated alone or in multiple instances with a singular platform (i.e., a base system 1504) to realize system 1500’s full operation. The modularity allows the clinical evaluation system 1500 to be designed to leverage the same synchronously acquired biophysical signals and data set and base platform to assess for the presence of several different diseases as such disease-specific algorithms are developed, thereby reducing testing and certification time and cost.

In various embodiments, different versions of the clinical evaluation system 1500 may implement the assessment system 103 (FIG. 1) by having included containing different feature computation modules that can be configured for a given disease state(s), medical condition(s), or indicating condition(s) of interest. In another embodiment, the clinical evaluation system 1500 may include more than one assessment system 103 and maybe selectively utilized to generate different scores specific to a classifier 116 of that engine 103. In this way, the modules of FIGS. 1 and 15 in a more general sense may be viewed as one configuration of a modular system in which different and/or multiple engines 103, with different and/or multiple corresponding classifiers 116, may be used depending on the configuration of module desired. As such, any number of embodiments of the modules of FIG. 1, with or without the respiration-rate specific feature(s), may exist.

In FIG. 15A, System 1500 can analyze one or more biophysical-signal data sets (e.g., 110) using machine-learned disease-specific algorithms to assess for the likelihood of elevated LVEDP, as one example, of pathology or abnormal state. System 1500 includes hardware and software components that are designed to work together in combination to facilitate the analysis and presentation of an estimation score using the algorithm to allow a physician to use that score, e.g., to assess for the presence or non-presence of a disease state, medical condition, or an indication of either.

The base system 1504 can provide a foundation of functions and instructions upon which each add-on module 1502 (which includes the disease-specific algorithm) then interfaces to assess for the pathology or indicating condition. The base system 1504, as shown in the example of FIG. 15A, includes a base analytical engine or analyzer 1506, a web-service data transfer API 1508 (shown as “DTAPI” 1508), a report database 1510, a web portal service module 1513, and the data repository 111 (shown as 112a).

Data repository 112a, which can be cloud-based, stores data from the signal capture system 102 (shown as 102b). Biophysical signal capture system 102b, in some embodiments, is a reusable device designed as a single unit with a seven-channel lead set and photoplethysmogram (PPG) sensor securely attached (i.e., not removable). Signal capture system 102b, together with its hardware, firmware, and software, provides a user interface to collect patient-specific metadata entered therein (e.g., name, gender, date of birth, medical record number, height, and weight, etc.) to synchronously acquire the patient’s electrical and hemodynamic signals. The signal capture system 102b may securely transmit the metadata and signal data as a single data package directly to the cloud-based data repository. The data repository 112a, in some embodiments, is a secure cloud-based database configured to accept and store the patient-specific data package and allow for its retrieval by the analytical engines or analyzer 1506 or 1514.

Base analytical engine or analyzer 1506 is a secure cloud-based processing tool that may perform quality assessments of the acquired signals (performed via “SQA” module 1516), the results of which can be communicated to the user at the point of care. The base analytical engine or analyzer 1506 may also perform pre-processing (shown via pre-processing module 1518) of the acquired biophysical signals (e.g., 110 - see FIG. 1). Web portal 1513 is a secure web-based portal designed to provide healthcare providers access to their patient’s reports. An example output of the web portal 1513 is shown by visualization 1536. The report databases (RD) 1512 is a secure database and may securely interface and communicate with other systems, such as a hospital or physician-hosted, remotely hosted, or remote electronic health records systems (e.g., Epic, Cerner, Allscrips, CureMD, Kareo, etc.) so that output score(s) (e.g., 118) and related information may be integrated into and saved with the patient’s general health record. In some embodiments, web portal 1513 is accessed by a call center to provide the output clinical information over a telephone. Database 1512 may be accessed by other systems that can generate a report to be delivered via the mail, courier service, personal delivery, etc.

Add-on module 1502 includes a second part 1514 (also referred to herein as the analytical engine (AE) or analyzer 1514 and shown as “AE add-on module” 1514) that operates with the base analytical engine (AE) or analyzer 1506. Analytical engine (AE) or analyzer 1514 can include the main function loop of a given disease-specific algorithm, e.g., the feature computation module 1520, the classifier model 1524 (shown as “Ensemble” module 1524), and the outlier assessment and rejection module 1524 (shown as “Outlier Detection” module 1524). In certain modular configurations, the analytical engines or analyzers (e.g., 1506 and 1514) may be implemented in a single analytical engine module.

The main function loop can include instructions to (i) validate the executing environment to ensure all required environment variables values are present and (ii) execute an analysis pipeline that analyzes a new signal capture data file comprising the acquired biophysical signals to calculate the patient’s score using the disease-specific algorithm. To execute the analysis pipeline, AE add-on module 1514 can include and execute instructions for the various feature modules 114 and classifier module 116 as described in relation to FIG. 1 to determine an output score (e.g., 118) of the metrics associated with the physiological state of a patient. The analysis pipeline in the AE add-on module 1514 can compute the features or parameters (shown as “Feature Computation” 1520) and identifies whether the computed features are outliers (shown as “Outlier Detection” 1522) by providing an outlier detection return for a signal-level response of outlier vs. non-outlier based on the feature. The outliers may be assessed with respect to the training data set used to establish the classifier (of module 116). AE add-on module 1514 may generate the patient’s output score (e.g., 118) (e.g., via classifier module 1524) using the computed values of the features and classifier models. In the example of an evaluation algorithm for the estimation of elevated LVEDP, the output score (e.g., 118) is an LVEDP score. For the estimation of CAD, the output score (e.g., 118) is a CAD score.

The clinical evaluation system 1500 can manage the data within and across components using the web-service DTAPIs 1508 (also may be referred to as HCPP web services in some embodiments). DTAPIs 1508 may be used to retrieve acquired biophysical data sets from, and to store signal quality analysis results to, the data repository 112a. DTAPIs 1508 may also be invoked to retrieve and provide the stored biophysical data files to the analytical engines or analyzers (e.g., 1506, 1514), and the results of the analytical engine’s analysis of the patient signals may be transferred using DTAPI 1508 to the report database 1510. DTAPIs 1508 may also be used, upon a request by a healthcare professional, to retrieve a given patient data set to the web portal module 1513, which may present a report to the healthcare practitioner for review and interpretation in a secure web-accessible interface.

Clinical evaluation system 1500 includes one or more feature libraries 1526 that store the respiration rate-related features 120 and various other features of the feature modules 122. The feature libraries 1526 may be a part of the add-on modules 1502 (as shown in FIG. 15A) or the base system 1504 (not shown) and are accessed, in some embodiments, by the AE add-on module 1514.

Further details of the modularity of modules and various configurations are provided in U.S. Provisional Pat. Application no. 63/235,960, filed Aug. 19, 2021, entitled “Modular Disease Assessment System,” which is hereby incorporated by reference herein in its entirety.

### Example Operation of the Modular Clinical Evaluation System

FIG. 15B shows a schematic diagram of the operation and workflow of the analytical engines or analyzers (e.g., 1506 and 1514) of the clinical evaluation system 1500 of FIG. 15A in accordance with an illustrative embodiment.

Signal quality assessment/rejection (1530). Referring to FIG. 15B, the base analytical engine or analyzer 1506 assesses (1530), via SQA module 1516, the quality of the acquired biophysical-signal data set while the analysis pipeline is executing. The results of the assessment (e.g., pass/fail) are immediately returned to the signal capture system’s user interface for reading by the user. Acquired signal data that meet the signal quality requirements are deemed acceptable (i.e., “pass”) and further processed and subjected to analysis for the presence of metrics associated with the pathology or indicating condition (e.g., elevated LVEDP or mPAP, CAD, PH/PAH, abnormal LVEF, HFpEF) by the AE add-on module 1514. Acquired signals deemed unacceptable are rejected (e.g., “fail”), and a notification is immediately sent to the user to inform the user to immediately obtain additional signals from the patient (see FIG. 2).

The base analytical engine or analyzer 1506 performs two sets of assessments for signal quality, one for the electrical signals and one for the hemodynamic signals. The electrical signal assessment (1530) confirms that the electrical signals are of sufficient length, that there is a lack of high-frequency noise (e.g., above 170 Hz), and that there is no power line noise from the environment. The hemodynamic signal assessment (1530) confirms that the percentage of outliers in the hemodynamic data set is below a pre-defined threshold and that the percentage and maximum duration that the signals of the hemodynamic data set are railed or saturated is below a pre-defined threshold.

Feature Value Computation (1532). The AE add-on module 1514 performs feature extraction and computation to calculate feature output values. In the example of the LVEDP algorithm, the AE add-on module 1514 determines, in some embodiments, a total of 446 feature outputs belonging to 18 different feature families (e.g., generated in modules 120 and 122), including the respiration rate-related features (e.g., generated in module 120). For the CAD algorithm, an example implementation of the AE add-on module 1514 determines a set of features, including 456 features corresponding to the same 18 feature families.

Additional descriptions of the various features, including those used in the LVEDP algorithm and other features and their feature families, are described in U.S. Provisional Pat. Application no. 63/235,960, filed Aug. 23, 2021, entitled “Method and System to Non-Invasively Assess Elevated Left Ventricular End-Diastolic Pressure”; U.S. Provisional Pat. Application no. 63/236,072, filed Aug. 23, 2021, entitled “Methods and Systems for Engineering Visual Features From Biophysical Signals for Use in Characterizing Physiological Systems”; U.S. Provisional Pat. Application no. 63/235,963, filed Aug. 23, 2021, entitled “Methods and Systems for Engineering Power Spectral Features From Biophysical Signals for Use in Characterizing Physiological Systems”; U.S. Provisional Pat. Application no. 63/235,968, filed Aug. 23, 2021, entitled “Methods and Systems for Engineering Wavelet-Based Features From Biophysical Signals for Use in Characterizing Physiological Systems”; U.S. Provisional Pat. Application no. 63/130,324, titled “Method and System to Assess Disease Using Cycle Variability Analysis of Cardiac and Photoplethysmographic Signals”; U.S. Provisional Patent Application no. 63/235,971, filed Aug. 23, 2021, entitled “Methods and Systems for Engineering photoplethysmographic Waveform Features for Use in Characterizing Physiological Systems”; U.S. Provisional Pat. Application no. 63/236,193, filed Aug. 23, 2021, entitled “Methods and Systems for Engineering Cardiac Waveform Features From Biophysical Signals for Use in Characterizing Physiological Systems”; U.S. Provisional Pat. Application no. 63/235,974, filed Aug. 23, 2021, entitled “Methods and Systems for Engineering Conduction Deviation Features From Biophysical Signals for Use in Characterizing Physiological Systems,” each of which is hereby incorporated by reference herein in its entirety.

Classifier Output Computation (1534). The AE add-on module 1514 then uses the calculated feature outputs in classifier models (e.g., machine-learned classifier models) to generate a set of model scores. The AE add-on module 1514 joins the set of model scores in an ensemble of the constituent models, which, in some embodiments, averages the output of the classifier models as shown in Equation 6 in the example of the LVEDP algorithm.

$$E n s e m b l e e s t i m a t i o n = \frac{M o d e {l}_{1} + M o d e {l}_{2} + … + M o d e {l}_{n}}{n}$$

In some embodiments, classifier models may include models that are developed based on ML techniques described in U.S. Pat. Publication No. 20190026430, entitled “Discovering Novel Features to Use in Machine Learning Techniques, such as Machine Learning Techniques for Diagnosing Medical Conditions”; or U.S. Pat. Publication No. 20190026431, entitled “Discovering Genomes to Use in Machine Learning Techniques,” each of which is hereby incorporated by reference herein in its entirety.

In the example of the LVEDP algorithm, thirteen (13) machine-learned classifier models are each calculated using the calculated feature outputs. The 13 classifier models include four ElasticNet machine-learned classifier models [9], four RandomForestClassifier machine-learned classifier models [10], and five extreme gradient boosting (XGB) classifier models [11]. In some embodiments, the patient’s metadata information, such as age, gender, and BMI value, may be used. The output of the ensemble estimation may be a continuous score. The score may be shifted to a threshold value of zero by subtracting the threshold value for presentation within the web portal. The threshold value may be selected as a trade-off between sensitivity and specificity. The threshold may be defined within the algorithm and used as the determination point for test positive (e.g., “Likely Elevated LVEDP”) and test negative (e.g., “Not Likely Elevated LVEDP”) conditions.

In some embodiments, the analytical engine or analyzer can fuse the set of model scores with a body mass index-based adjustment or an adjustment based on age or gender. For example, the analytical engine or analyzer can average the model estimation with a sigmoid function of the patient BMI having the form sigmoid (x) =

Physician Portal Visualization (1536). The patient’s report may include a visualization 1536 of the acquired patient data and signals and the results of the disease analyses. The analyses are presented, in some embodiments, in multiple views in the report. In the example shown in FIG. 15B, the visualization 1536 includes a score summary section 1540 (shown as “Patient LVEDP Score Summary” section 1540), a threshold section 1542 (shown as “LVEDP Threshold Statistics” section 1542), and a frequency distribution section 1544 (shown as “Frequency Distribution” section 1508). A healthcare provider, e.g., a physician, can review the report and interpret it to provide a diagnosis of the disease or to generate a treatment plan.

The healthcare portal may list a report for a patient if a given patient’s acquired signal data set meets the signal quality standard. The report may indicate a disease-specific result (e.g., elevated LVEDP) being available if the signal analysis could be performed. The patient’s estimated score (shown via visual elements 118a, 118b, 118c) for the disease-specific analysis may be interpreted relative to an established threshold.

In the score summary section 1540 shown in the example of FIG. 15B, the patient’s score 118a and associated threshold are superimposed on a two-tone color bar (e.g., shown in section 1540) with the threshold located at the center of the bar with a defined value of “0” representing the delineation between test positive and test negative. The left of the threshold may be lightly shaded light and indicates a negative test result (e.g., “Not Likely Elevated LVEDP”) while to the right of the threshold may be darkly shaded to indicate a positive test result (e.g., “Likely Elevated LVEDP”).

The threshold section 1542 shows reported statistics of the threshold as provided to a validation population that defines the sensitivity and specificity for the estimation of the patient score (e.g., 118). The threshold is the same for every test regardless of the individual patient’s score (e.g., 118), meaning that every score, positive or negative, may be interpreted for accuracy in view of the provided sensitivity and specificity information. The score may change for a given disease-specific analysis as well with the updating of the clinical evaluation.

The frequency distribution section 1544 illustrates the distribution of all patients in two validation populations (e.g., (i) a non-elevated population to indicate the likelihood of a false positive estimation and (ii) an elevated population to indicate a likelihood of a false negative estimation). The graphs (1546, 1548) are presented as smooth histograms to provide context for interpreting the patient’s score 118 (e.g., 118b, 118c) relative to the test performance validation population patients.

The frequency distribution section 1540 includes a first graph 1546 (shown as “Non-Elevated LVEDP Population” 1546) that shows the score (118b), indicating the likelihood of the non-presence of the disease, condition, or indication, within a distribution of a validation population having non-presence of that disease, condition, or indication and a second graph 1548 (shown as “Elevated LVEDP Population” 1548) that shows the score (118c), indicates the likelihood of the presence of the disease, condition, or indication, within a distribution of validation population having the presence of that disease, condition, or indication. In the example of the assessment of elevated LVDEP, the first graph 1546 shows a non-elevated LVEDP distribution of the validation population that identifies the true negative (TN) and false positive (FP) areas. The second graph 1548 shows an elevated LVEDP distribution of the validation population that identifies the false negative (TN) and true positive (FP) areas.

The frequency distribution section 1540 also includes interpretative text of the patient’s score relative to other patients in a validation population group (as a percentage). In this example, the patient has an LVEDP score of -0.08, which is located to the left side of the LVEDP threshold, indicating that the patient has “Not Likely Elevated LVEDP.”

The report may be presented in the healthcare portal, e.g., to be used by a physician or healthcare provider in their diagnosis for indications of left-heart failure. The indications include, in some embodiments, a probability or a severity score for the presence of a disease, medical condition, or an indication of either.

Outlier Assessment and Rejection Detection (1538). Following the AE add-on module 1514 computing the feature value outputs (in process 1532) and prior to their application to the classifier models (in process 1534), the AE add-on module 1514 is configured in some embodiments to perform outlier analysis (shown in process 1538) of the feature value outputs. Outlier analysis evaluation process 1538 executes a machine-learned outlier detection module (ODM), in some embodiments, to identify and exclude anomalous acquired biophysical signals by identifying and excluding anomalous feature output values in reference to the feature values generated from the validation and training data. The outlier detection module assesses for outliers that present themselves within sparse clusters at isolated regions that are out of distribution from the rest of the observations. Process 1538 can reduce the risk that outlier signals are inappropriately applied to the classifier models and produce inaccurate evaluations to be viewed by the patient or healthcare provider. The accuracy of the outlier module has been verified using hold-out validation sets in which the ODM is able to identify all the labeled outliers in a test set with the acceptable outlier detection rate (ODR) generalization.

While the methods and systems have been described in connection with certain embodiments and specific examples, it is not intended that the scope be limited to the particular embodiments set forth, as the embodiments herein are intended in all respects to be illustrative rather than restrictive. The respiration rate-related features discussed herein may ultimately be employed to make, or to assist a physician or other healthcare provider in making, noninvasive diagnoses or determinations of the presence or non-presence and/or severity of other diseases, medical conditions, or indication of either, such as, e.g., coronary artery disease, pulmonary hypertension and other pathologies as described herein using similar or other development approaches. In addition, the example analysis, including the respiration rate-related features, can be used in the diagnosis and treatment of other cardiac-related pathologies and indicating conditions as well as neurological-related pathologies and indicating conditions, such assessment can be applied to the diagnosis and treatment (including, surgical, minimally invasive, and/or pharmacologic treatment) of any pathologies or indicating conditions in which a biophysical signal is involved in any relevant system of a living body. One example in the cardiac context is the diagnosis of CAD, and other diseases, medical conditions, or indicating conditions disclosed herein and its treatment by any number of therapies, alone or in combination, such as the placement of a stent in a coronary artery, the performance of an atherectomy, angioplasty, prescription of drug therapy, and/or the prescription of exercise, nutritional and other lifestyle changes, etc. Other cardiac-related pathologies or indicating conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other diseases such as blood or other disorders), as well as other cardiac-related pathologies, indicating conditions and/or diseases. Non-limiting examples of neurological-related diseases, pathologies or indicating conditions that may be diagnosed include, e.g., epilepsy, schizophrenia, Parkinson’s Disease, Alzheimer’s Disease (and all other forms of dementia), autism spectrum (including Asperger syndrome), attention deficit hyperactivity disorder, Huntington’s Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, indicating conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/indicating conditions and vision-related diseases/indicating conditions.

In addition, the clinical evaluation system described herein may be configured to analyze biophysical signals such as an electrocardiogram (ECG), electroencephalogram (EEG), gamma synchrony, respiratory function signals, pulse oximetry signals, perfusion data signals; quasi-periodic biological signals, fetal ECG signals, blood pressure signals; cardiac magnetic field signals, heart rate signals, among others.

Further examples of processing that may be used with the exemplified method and system disclosed herein are described in: U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Pat. Publication Nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication Nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. Pat. Application Nos. 16/831,264; 16/831,380; 17/132869; PCT Application Nos. PCT/IB2020/052889; PCT/IB2020/052890, each of which is hereby incorporated by reference herein in its entirety.

The following patents, applications, and publications as listed below and throughout this document are hereby incorporated by reference in their entirety herein.

### LIST OF REFERENCES

[1] P. H. Charlton et al., “Breathing Rate Estimation from the Electrocardiogram and Photoplethysmogram: A Review,” IEEE Rev. Biomed. Eng., vol. 11, pp. 2-20, October 2018.
[2] P. H. Charlton, T. Bonnici, L. Tarassenko, D. A. Clifton, R. Beale, and P. J. Watkinson, “An assessment of algorithms to estimate respiratory rate from the electrocardiogram and photoplethysmogram,” Physiol. Meas., vol. 37, no. 4, pp. 610-626, March 2016.
[3] A. Garde, W. Karlen, J. M. Ansermino, and G. A. Dumont, “Estimating respiratory and heart rates from the correntropy spectral density of the photoplethysmogram,” PLoS One, vol. 9, no. 1, January 2014.
[4] D. J. Meredith, D. Clifton, P. Charlton, J. Brooks, C. W. Pugh, and L. Tarassenko, “Photoplethysmographic derivation of respiratory rate: A review of relevant physiology,” Journal of Medical Engineering and Technology, vol. 36, no. 1. pp. 1-7, Jan-2012.
[5] M. A. F. Pimentel et al., “Toward a robust estimation of respiratory rate from pulse oximeters,” IEEE Trans. Biomed. Eng., vol. 64, no. 8, pp. 1914-1923, August 2017.
[6] M. A. Russo, D. M. Santarelli, and D. O'Rourke, “The physiological effects of slow breathing in the healthy human,” Breathe, vol. 13, no. 4. European Respiratory Society, pp. 298-309, 01-Dec-2017.
[7] C. M. Bishop, Pattern Recognition and Machine Learning (Information Science and Statistics). Secaucus, NJ, USA: Springer-Verlag New York, Inc., 2006.
[8] I. Tolstikhin, B. K. Sriperumbudur, and B. Schölkopf, “Minimax Estimation of Maximum Mean Discrepancy with Radial Kernels.”
[9] H. Zou and T. Hastie, “Regularization and variable selection via the elastic net,”, Journal of the Roy. Statist. Soc., ser. B, vol. 67, no. 2, pp.301-320, 2005.
[10] Breiman, “Random Forests,” Machine Learning, 45(1), 5-32, 2001.
[11] Chen, Tianqi; Guestrin, Carlos (2016). “XGBoost: A Scalable Tree Boosting System”. In Krishnapuram, Balaji; Shah, Mohak; Smola, Alexander J.; Aggarwal, Charu C.; Shen, Dou; Rastogi, Rajeev (eds.). Proceedings of the 22nd ACM SIGKDD International Conference on Knowledge Discovery and Data Mining, San Francisco, CA, USA, August 13-17, 2016. ACM. pp. 785-794.
[12] Tompkins W.J., Pan J., A Real-Time QRS Detection Algorithm. IEEE Transactions on Biomedical Engineering, BME-32(3) pages 230-236 (1985).
[13] Karlen W, Ansermino J M and Dumont G, Adaptive pulse segmentation and artifact detection in photoplethysmography for mobile applications Conf. Proc. IEEE Engineering in Medicine and Biology Society pp 3131-4 (2012).

1. A method for non-invasively estimating values of one or more metrics associated with a disease state, medical condition, or indication of either, the method comprising:
acquiring, by one or more processors, a biophysical-signal data set of a subject comprising one or more first biophysical signals and one or more second biophysical signals, wherein the one or more first biophysical signals are simultaneously acquired with respect to the one or more second biophysical signals;
determining, by the one or more processors, values of respiration rate-related features that describe one or more respiration associated properties or one or more heart rate variability-associated properties, wherein the determination is based on the one or more first biophysical signals and the one or more second biophysical signals; and
determining, by the one or more processors, an estimated value for presence of a metric associated with the disease state, medical condition or indication of either based on an application of the determined values of the respiration rate-related features to an estimation model,
wherein the estimated value for the presence of the metric is used in the estimation model to i) non-invasively estimate or indicate the presence of the disease state, medical condition or indication of either for use in a diagnosis, or to direct treatment, of the disease state, medical condition or indication of either.

2. The method of claim 1 further comprising:
outputting, by the one or more processors, the values of the respiration rate-related features.

3. The method of claim 1, wherein the one or more first biophysical signals comprise biopotential signals acquired for three channels of measurements.

4. The method of claim 1, wherein the one or more second biophysical signals comprise photoplethysmographic signals acquired from optical sensors.

5. The method of claim 1, wherein the biophysical-signal data set comprises (i) biopotential signals acquired for three channels of measurements and (ii) photoplethysmographic signals acquired from optical sensors.

6. The method of claim 1, wherein the step of determining the values of the heart rate variability-associated properties comprises:
generating, by the one or more processors, via a modulation operator, a modulation data set of the biophysical-signal data set, wherein the modulation operator is selected from the group consisting of an amplitude modulation operator, a frequency modulation operator, a peak modulation operator, an amplitude continuous modulation operator, a frequency continuous modulation operator, and an adaptive filter; and
determining, by the one or more processors, one or more values of features extracted from the modulation data set, wherein the one or more features include a feature associated with heart rate variability.

7. The method of claim 6, wherein the feature associated with heart rate variability is determined as a statistical assessment of frequency-modulated data generated by the frequency modulation operator or frequency continuous modulation operator performed on a signal of the biophysical-signal data set.

8. The method of claim 1, wherein the step of determining the values of the one or more respiration associated properties comprises:
generating, by the one or more processors, via a modulation operator, a modulation data set of the biophysical-signal data set, wherein the modulation operator is selected from the group consisting of an amplitude modulation operator, a frequency modulation operator, a peak modulation operator, an amplitude continuous modulation operator, a frequency continuous modulation operator, and an adaptive filter;
generating, by the one or more processors, one or more respiration rate estimations using the modulation data set; and
determining, by the one or more processors, one or more values of features extracted from the one or more respiration rate estimations, wherein the one or more features include a feature associated with a statistical assessment of the one or more respiration rate estimations.

9. The method of claim 1, wherein the step of determining the values of the one or more respiration associated properties comprises:
generating, by the one or more processors, one or more relative entropy estimations using the modulation data set; and
determining, by the one or more processors, one or more values of features extracted from the one or more relative entropy estimations, wherein the one or more features include a feature associated with a statistical assessment of the one or more relative entropy estimations.

10. The method of claim 1, wherein the step of determining the values of the one or more respiration associated properties comprises:
generating, by the one or more processors, one or more maximum mean discrepancy (MMD) distance metric using the modulation data set; and
determining, by the one or more processors, one or more values of features extracted from the one or more maximum mean discrepancy (MMD) distance metric, wherein the one or more features includes a feature associated with a statistical assessment of the one or more maximum mean discrepancy distance metric.

11. The method of claim 1, wherein the step of determining the values of the one or more respiration associated properties comprises:
generating, by the one or more processors, one or more coherence metrics using the modulation data set and a proxy respiration waveform generated from a determined respiration rate; and
determining, by the one or more processors, one or more values of features extracted from the one or more coherence metric, wherein the one or more features include a feature associated with a statistical assessment of the one or more coherence metric.

12. The method of claim 1 further comprising:
causing, by the one or more processors, generation of a visualization of the estimated value for the presence of the disease state, medical condition or indication of either, wherein the generated visualization is rendered and displayed at a display of a computing device and/or presented in a report.

13. The method of claim 1, wherein the values of the one or more respiration associated properties or the heart rate variability-associated properties are used in the model selected from the group consisting of a linear model, a decision tree model, a support vector machine model, and a neural network model.

14. The method of claim 13, wherein the model further includes features selected from the group consisting of:
one or more depolarization or repolarization wave propagation associated features;
one or more depolarization wave propagation deviation associated features;
one or more cycle variability associated features;
one or more dynamical system associated features;
one or more cardiac waveform topologic and variations associated features;
one or more PPG waveform topologic and variations associated features;
one or more cardiac or PPG signal power spectral density associated features;
one or more cardiac or PPG signal visual associated features; and
one or more predictability features.

15. The method of claim 1, wherein the disease state, medical condition or indication of either is selected from the group consisting of coronary artery disease, pulmonary hypertension, pulmonary arterial hypertension, pulmonary hypertension due to left heart disease, rare disorders that lead to pulmonary hypertension, left ventricular heart failure or left-sided heart failure, right ventricular heart failure or right-sided heart failure, systolic heart failure, diastolic heart failure, ischemic heart disease, and arrhythmia.

16. The method of claim 1, further comprising:
acquiring, by one or more acquisition circuits of a measurement system, voltage gradient signals over the one or more channels, wherein the voltage gradient signals are acquired at a frequency greater than about 1 kHz; and
generating, by the one or more acquisition circuits, the obtained biophysical data set from the acquired voltage gradient signals.

17. The method of claim 1 further comprising:
acquiring, by one or more acquisition circuits of a measurement system, one or more photoplethysmographic signals; and
generating, by the one or more acquisition circuits, the obtained biophysical data set from the acquired voltage gradient signals.

18. The method of claim 1, wherein the one or more processors are located in a cloud platform or a local computing device.

19. A system comprising:
a processor; and
a memory having instructions stored thereon, wherein execution of the instructions by the processor causes the processor to:
acquire a biophysical-signal data set of a subject comprising one or more first biophysical signals and one or more second biophysical signals, wherein the one or more first biophysical signals are simultaneously acquired with respect to the one or more second biophysical signals;
determine values of respiration rate-related features that describe one or more respiration associated properties or one or more heart rate variability-associated properties, wherein the determination is based on the one or more first biophysical signals and the one or more second biophysical signals; and
determine an estimated value for presence of a metric associated with the disease state, medical condition or indication of either based on an application of the determined values of the respiration rate-related features to an estimation model,
wherein the estimated value for the presence of the metric is used in the estimation model to i) non-invasively estimate or indicate the presence of the disease state, medical condition or indication of either for use in a diagnosis, or to direct treatment, of the disease state, medical condition or indication of either.

20. A non-transitory computer-readable medium having instructions stored thereon, wherein execution of the instructions by a processor causes the processor to:
acquire a biophysical-signal data set of a subject comprising one or more first biophysical signals and one or more second biophysical signals, wherein the one or more first biophysical signals are simultaneously acquired with respect to the one or more second biophysical signals;
determine values of respiration rate-related features that describe one or more respiration associated properties or one or more heart rate variability-associated properties, wherein the determination is based on the one or more first biophysical signals and the one or more second biophysical signals; and
determine an estimated value for presence of a metric associated with the disease state, medical condition or indication of either based on an application of the determined values of the respiration rate-related features to an estimation model,
wherein the estimated value for the presence of the metric is used in the estimation model to i) non-invasively estimate or indicate the presence of the disease state, medical condition or indication of either for use in a diagnosis, or to direct treatment, of the disease state, medical condition or indication of either.
